(19) United States
(12) Patent Application Publication
(10) Pub. No.: US 2024/0239741 A1
(43) Pub. Date: Jul. 18, 2024

(54) META-ESTER AROMATIC DIAMINES, METHOD FOR PRODUCING SAME, AND POLYIMIDE HAVING SAID META-ESTER AROMATIC DIAMINES AS RAW MATERIAL

(71) Applicant: SEIKA CORPORATION, Wakayama (JP)

(72) Inventors: Mitsutaka Imoto, Wakayama (JP); Yasuyuki Miyata, Wakayama (JP); Motonori Takeda, Wakayama (JP); Kazuhide Nishiyama, Wakayama (JP); Hitoshi Yamato, Wakayama (JP)

(21) Appl. No.: 18/558,803

(22) PCT Filed: Mar. 25, 2022
(86) PCT No.: PCT/JP2022/014457

§ 371 Date: Nov. 3, 2023

(30) Foreign Application Priority Data
May 14, 2021 (JP) 2021-082154
Mar. 23, 2022 (JP) 2022-047016

(51) Int. Cl. C07C 317/22; C07C 229/60; C08G 73/10

(52) U.S. Cl. CPC C07C 317/22; C07C 229/60; C08G 73/10; C08G 73/1007

(57) ABSTRACT

[Problems] The purposes of the present invention are to provide a novel meta-type ester-containing aromatic diamines, a method for preparing the same, and a polyimide synthesis.

[Solution] The present invention provides a compound represented by the following formula (1) and a method for preparing the same:

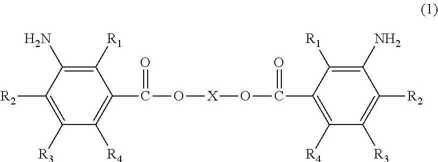

In the formula (1), X is selected from the following structures (a), (b) and (c),

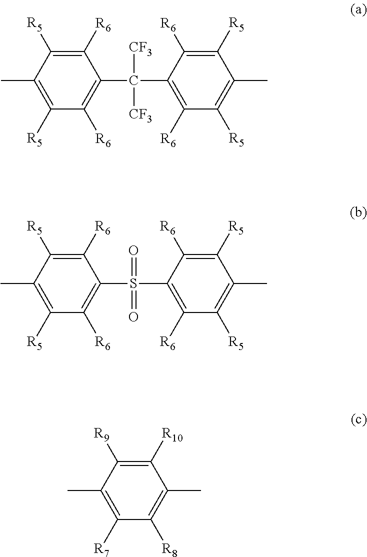

wherein R₁, R₂, R₃, and R₄ in the formula (1) and R₅, R₆, R₇, R₈, R₉ and R₁₀ in (a), (b) and (c) are, independently of each other, a hydrogen atom, an optionally substituted alkyl group having 1 to 6 carbon atoms, or an alkoxy group having 1 to 3 carbon atoms, provided that at least one of R₇, R₈, R₉ and R₁₀ is the alkyl group or the alkoxy group.

### TECHNICAL FIELD

The present invention relates to meta-type ester-containing aromatic diamines and derivatives thereof effective as a raw material for highly-functional polymers, including polyimide, and various kinds of organic compounds, and a method for preparing the same.

### BACKGROUND ART

A high-speed and large-capacity communication is required for a printed wiring board and the like used in the information communication field and, thus, it is expected to use a frequency band higher than conventionally used. Achieving a high frequency, however, causes a problem of increased transmission loss. The transmission loss is divided into contributions of resistive loss and dielectric loss. Among them, the resistive loss has a feature of turning into heat in proportion to a frequency, and the dielectric loss has a feature of being proportionate to a frequency, a dielectric loss tangent, and a relative dielectric constant.

Materials that can withstand usages in a high frequency band need to have excellent electric properties, in particular, a low dielectric constant and a low dielectric loss tangent, in addition to a heat resistance. There are known, for example, a polyimide resin (PI) and a polyamide resin as highly heat-resistant materials (Non-Patent Documents 1 and 2). These resins, however, have an imide group or amide group structure, which is high in polarity, in the molecules, and their contributions usually cause most of PI to have a dielectric constant (k) exceeding 3.0. There is also known, for example, a polyesterimide resin (PEI) as a PI material with excellent electric properties (Non-Patent Document 3). It, however, has problems, such as poor thermoplasticity, poor fluidity when molten, poor solubility in a solvent, and poor workability.
Non-Patent Document 1: Pathrick R. A. et al. “Journal of Applied Polymer Science”, vol. 132, p. 41684 to 41692, 2015.
Non-Patent Document 2: Akhter Z. et al. “Polymer Bulletin” vol. 74, p. 3889 to 3906, 2017.
Non-Patent Document 3: Masatoshi Hasegawa. et al. “Polymers”, vol. 12, p. 859, 2020.
Non-Patent Document 4: S. Tamai et al. “Polymer”, vol. 37, p. 3683 to 3692, 1996.

### DISCLOSURE OF THE INVENTION

### Problems to Be Solved by the Invention

There is proposed PI with lowered dielectric constant as a material with excellent high heat resistance and electric properties. The PI is an attractive material for molecular design for lowering dielectric constant in terms of diversity in designing a diamine as its monomer. The basic concept of lowering dielectric constant of the PI lies in how to dilute (reduce) an imide group concentration that contributes to high dielectric constant. In order to lower the imide group concentration in the PI, it is effective to employ a diamine having aromatic rings with three or more nuclei instead of a binuclear like oxydianiline, which is a typical aromatic diamine. Furthermore, introducing an ester part in a PI main chain is effective for reducing moisture absorbency of the PI and lowering the dielectric constant (Non-Patent Document 3). The aromatic diamine described in Non-Patent Document 3, however, impairs the workability of the PI resin as the linearity of the PI main chain improves. In order to improve the workability of the PI, it is effective to use a meta-type aromatic diamine as a raw material (Non-Patent Document 4), but it does not contribute to the lowering of the dielectric constant of the PI.

Therefore, in order to achieve excellent high heat resistance, electric properties, and workability at the same time, it is effective to use a meta-type ether-based aromatic diamine as a raw material of the PI. To produce meta-type ether-based aromatic diamine precursors, however, it requires severe reactive conditions of 145 to 150° C./5 hours, and further, 170 to 180° C./18 hours (Non-Patent Document 4). On the other hand, ester-based aromatic diamine precursors can be synthesized in a calm reactive condition of room temperature/12 hours. In view of the above-described circumstances, one of its objectives of the present invention is to provide easily producible meta-type ester-containing aromatic diamine compounds and derivatives thereof effective as a resin material, such as a polyimide resin, an electronic material, or their intermediate or a raw material, and a method for preparing the same.

### Solutions to the Problems

The inventors have reached to form the present invention as the result of intensive studies on the problems of aromatic diamines as described above by preparing new meta-type ester-containing aromatic diamines as a trinuclear or tetranuclear bis (3-aminobenzoyloxy) compound having 3-aminobenzoyloxy, and a pentanuclear meta-type ester-containing aromatic diamine.

That is, the present invention provides a compound represented by the following formula (1) and a method for preparing the same:

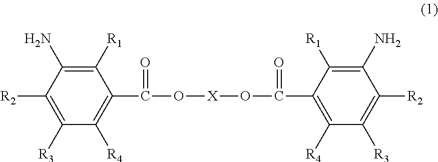

In the formula (1), X is selected from the following structures (a), (b) and (c),

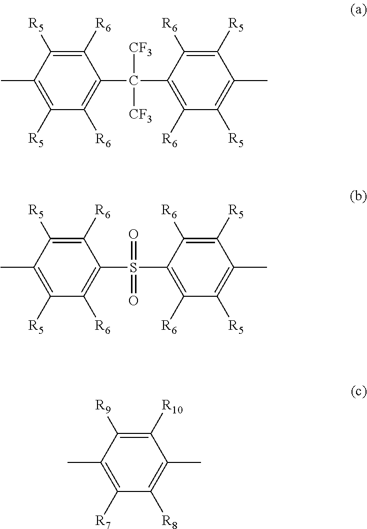

wherein R₁, R₂, R₃, and R₄ in the formula (1) and R₅, R₆, R₇, R₈, R₉ and R₁₀ in (a), (b) and (c) are, independently of each other, a hydrogen atom, an optionally substituted alkyl group having 1 to 6 carbon atoms, or an alkoxy group having 1 to 3 carbon atoms, provided that at least one of R₇, R₈, R₉ and R₁₀ is the alkyl group or the alkoxy group.

The present invention also provides a compound represented by the following formula (1′) and a method for preparing the same:

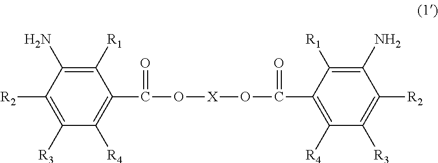

In the formula (1′), X is the following structure (d),

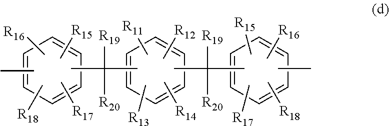

wherein R₁, R₂, R₃, R₄, R₁, R₁₂, R₁₃, R₁₄, R₁₅, R₁₆, R₁₇, R₁₈, R₁₉ and R₂₀ are, independently of each other, a hydrogen atom, an optionally substituted alkyl group having 1 to 6 carbon atoms, or an alkoxy group having 1 to 3 carbon atoms.

Furthermore, the present invention provides a polyimide compound as a reaction product obtained by reacting the diamine compound, an acid anhydride, and optional other diamine compound.

### Effects of the Invention

The meta-type ester-containing aromatic diamine of the present invention is excellent in solubility in various kinds of solvents. The meta-type ester-containing aromatic diamine of the present invention has three or more aromatic ring nuclei, so that an imide concentration of the obtained polyimide is capable of being lower. The meta-type ester-containing aromatic diamine of the present invention has an ester part, so that a moisture absorbency of the obtained polyimide is capable of being lower. Therefore, the present meta-type ester-containing aromatic diamine is effective for lowering a dielectric constant of the polyimide. Furthermore, the ester-containing aromatic diamine of the present invention is of a meta-type, which is preferably usable as a polyimide material having better workability.

### BRIEF DESCRIPTION OF THE DRAWINGS

FIG. 1 is a chart of a ¹H-NMR spectrum of a compound prepared in Example 2.

FIG. 2 is an enlarged chart of the ¹H-NMR spectrum of the compound prepared in Example 2.

FIG. 3 is a chart of a ¹³C-NMR spectrum of the compound prepared in Example 2.

FIG. 4 is an enlarged chart of the ¹³C-NMR spectrum of the compound prepared in Example 2.

FIG. 5 is a chart of a ¹H-NMR spectrum of a compound prepared in Example 4.

FIG. 6 is an enlarged chart of the ¹H-NMR spectrum of the compound prepared in Example 4.

FIG. 7 is a chart of a ¹³C-NMR spectrum of the compound prepared in Example 4.

FIG. 8 is an enlarged chart of the ¹³C-NMR spectrum of the compound prepared in Example 4.

FIG. 9 is a chart of a ¹H-NMR spectrum of a compound prepared in Example 6.

FIG. 10 is an enlarged chart of the ¹H-NMR spectrum of the compound prepared in Example 6.

FIG. 11 is a chart of a ¹³C-NMR spectrum of the compound prepared in Example 6.

FIG. 12 is an enlarged chart of the ¹³C-NMR spectrum of the compound prepared in Example 6.

FIG. 13 is a chart of a ¹H-NMR spectrum of a compound prepared in Example 8.

FIG. 14 is an enlarged chart of the ¹H-NMR spectrum of the compound prepared in Example 8.

FIG. 15 is a chart of a ¹³C-NMR spectrum of the compound prepared in Example 8.

FIG. 16 is an enlarged chart of the ¹³C-NMR spectrum of the compound prepared in Example 8.

FIG. 17 is an FT-IR spectrum of polyamide acid prepared in Example 9.

FIG. 18 is an FT-IR spectrum of a polyimide powder prepared in Example 9.

FIG. 19 is an FT-IR spectrum of a polyimide powder prepared in Example 10.

FIG. 20 is an FT-IR spectrum of a polyimide powder prepared in Example 11.

FIG. 21 is an FT-IR spectrum of a polyimide powder prepared in Example 12.

FIG. 22 is an FT-IR spectrum of a polyimide powder prepared in Example 13.

FIG. 23 is an FT-IR spectrum of a polyimide powder prepared in Example 14.

FIG. 24 is a chart of a ¹H-NMR spectrum of a compound prepared in Example 9.

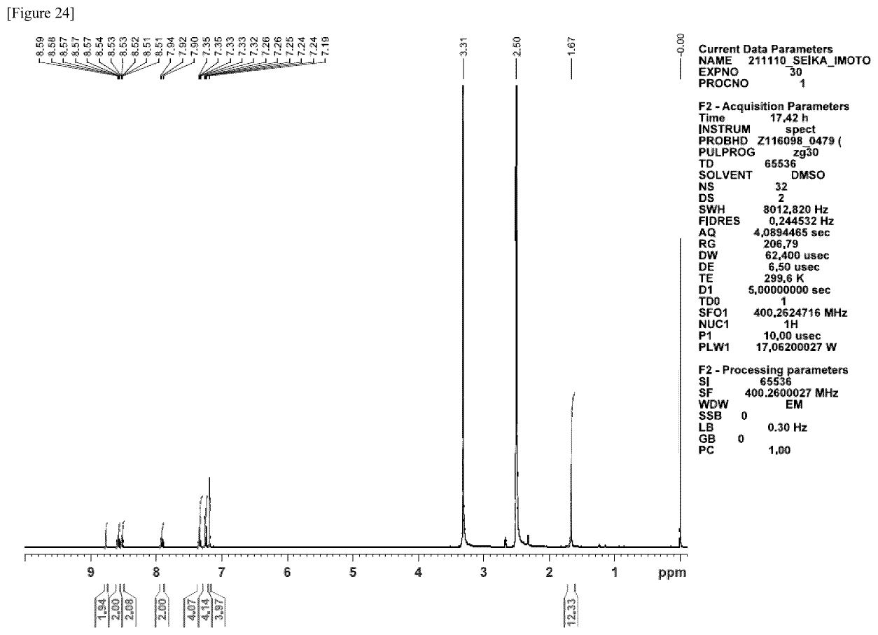

FIG. 25 is an enlarged chart of the ¹H-NMR spectrum of the compound prepared in Example 9.

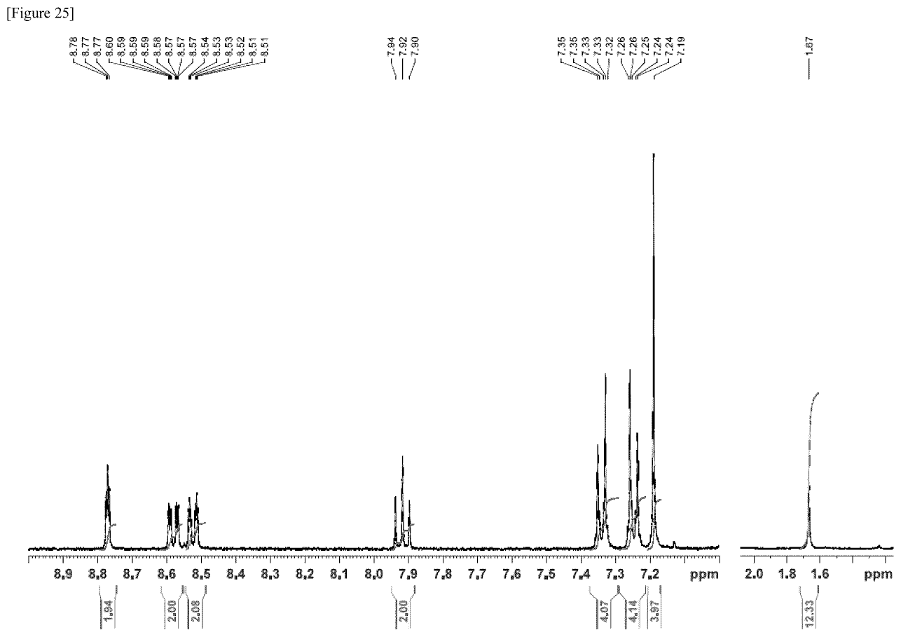

FIG. 26 is a chart of a ¹H-NMR spectrum of a compound prepared in Example 10.

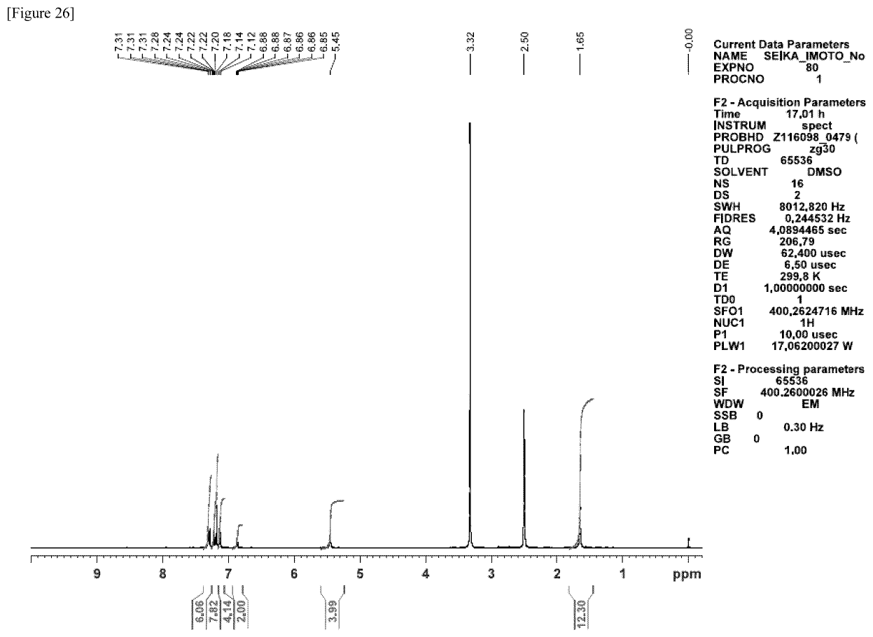

FIG. 27 is an enlarged chart of the ¹H-NMR spectrum of the compound prepared in Example 10.

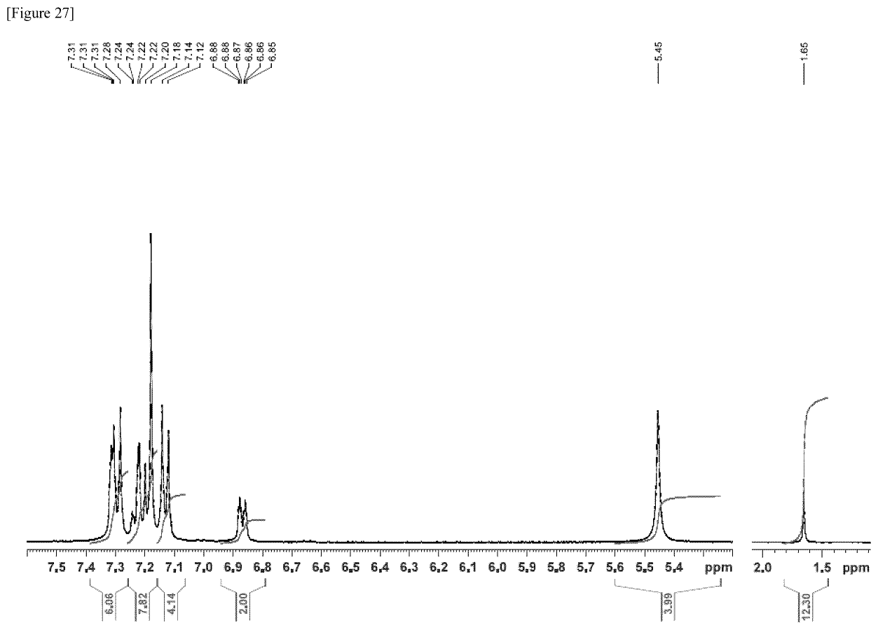

FIG. 28 is a chart of a ¹³C-NMR spectrum of the compound prepared in Example 10.

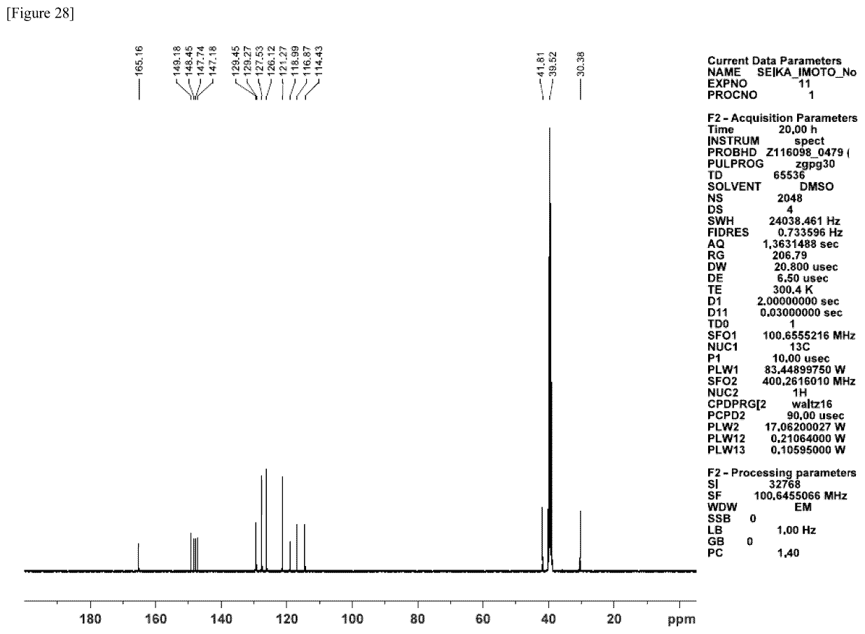

FIG. 29 is an enlarged chart of the ¹³C-NMR spectrum of the compound prepared in Example 10.

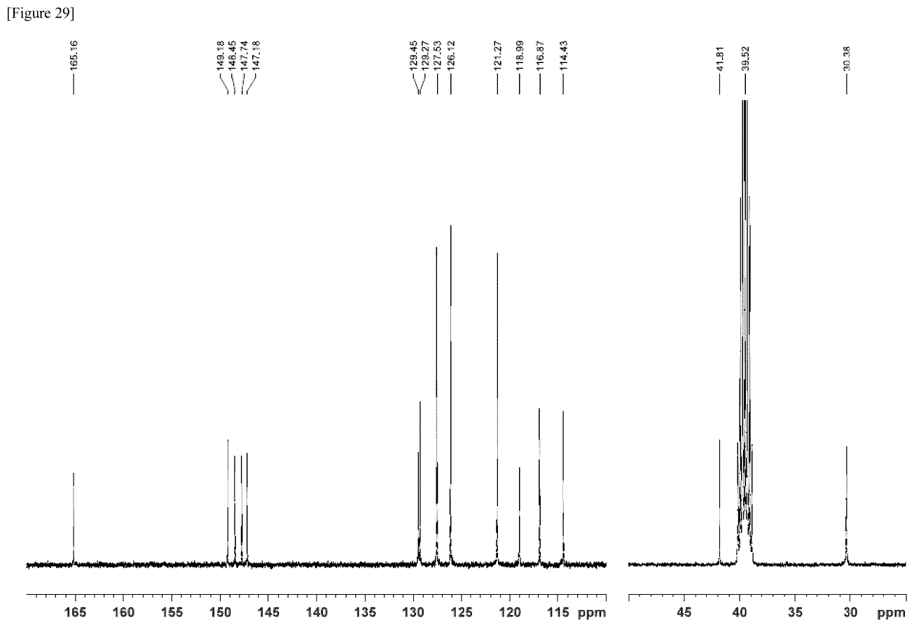

FIG. 30 is a chart of a ¹H-NMR spectrum of a compound prepared in Example 11.

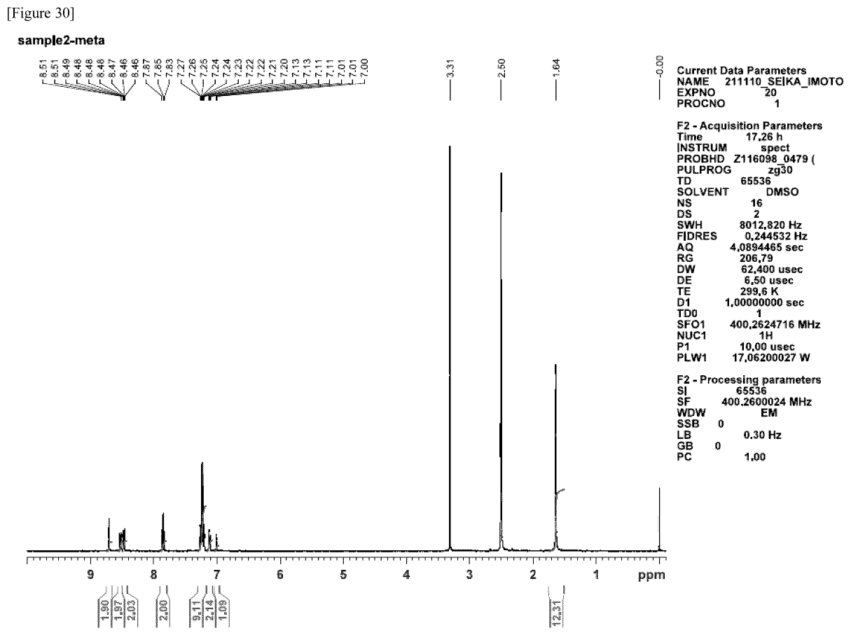

FIG. 31 is an enlarged chart of the ¹H-NMR spectrum of the compound prepared in Example 11

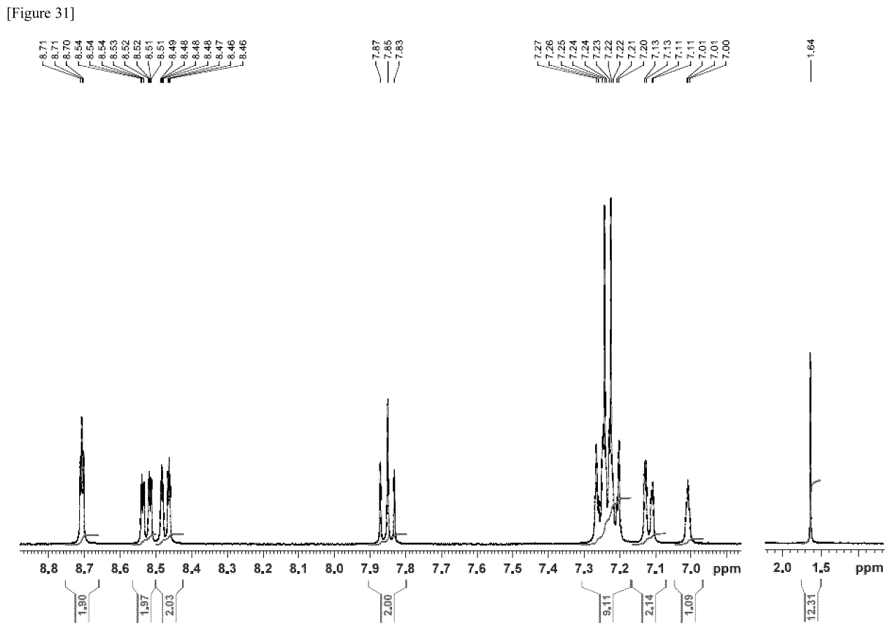

FIG. 32 is a chart of a ¹H-NMR spectrum of a compound prepared in Example 11

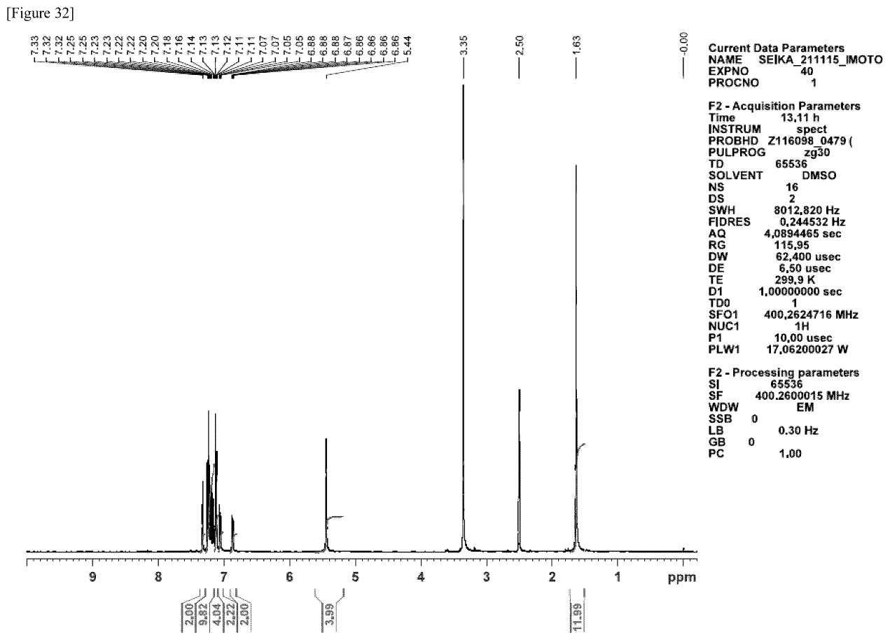

FIG. 33 is an enlarged chart of the ¹H-NMR spectrum of the compound prepared in Example 11.

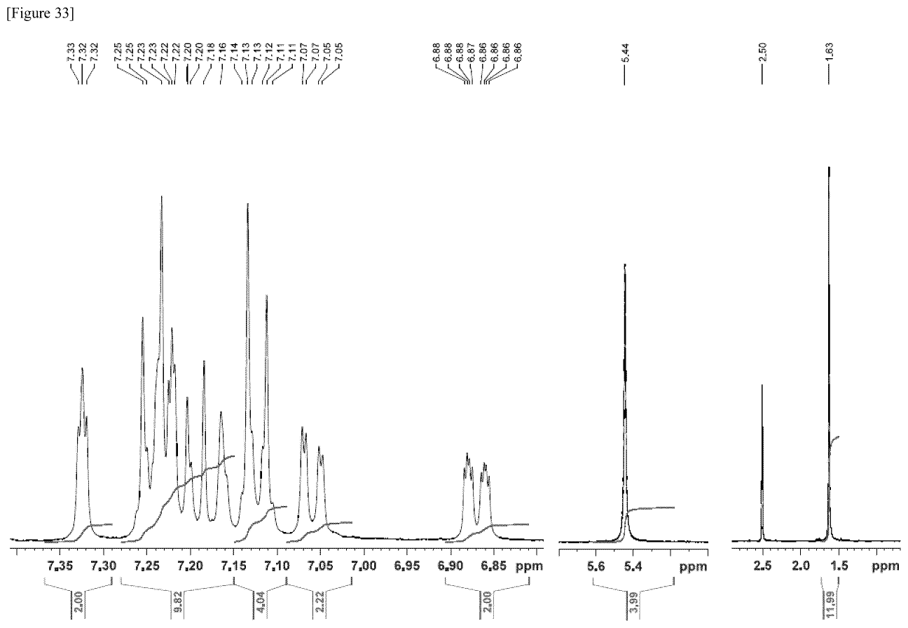

FIG. 34 is a chart of a ¹³C-NMR spectrum of the compound prepared in Example 11.

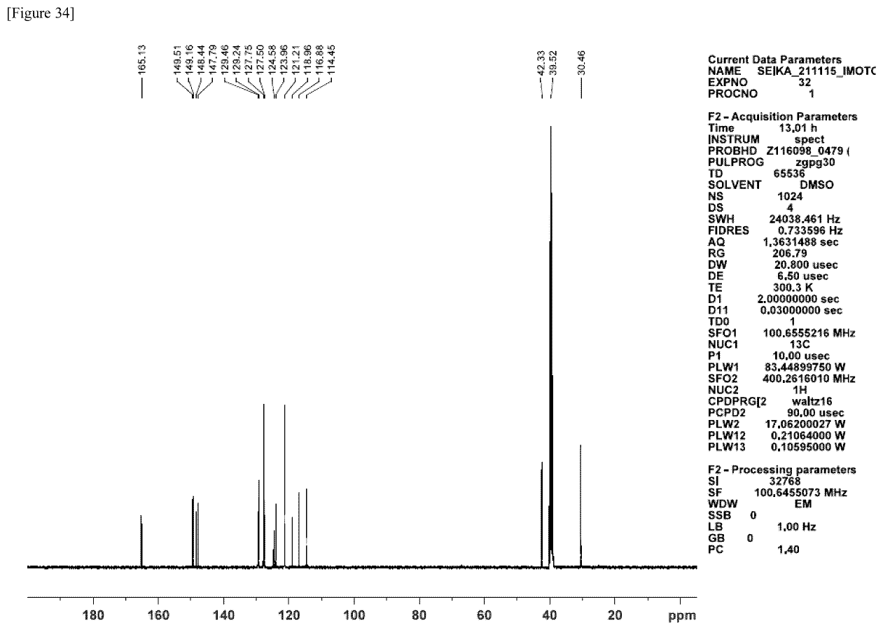

FIG. 35 is an enlarged chart of the ¹³C-NMR spectrum of the compound prepared in Example 11.

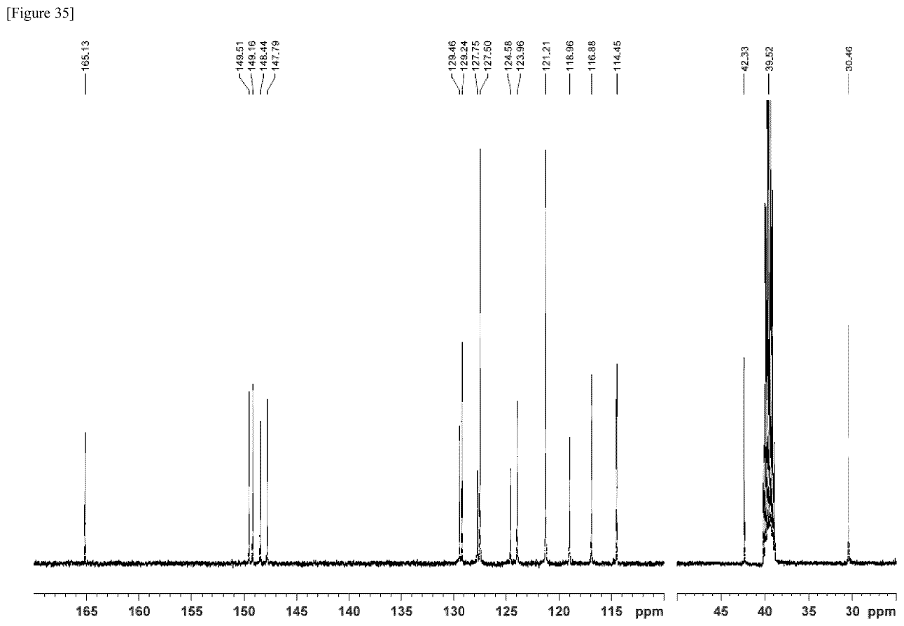

### DESCRIPTION OF PREFERRED EMBODIMENTS

One aspect of the present invention relates to a meta-type ester-containing aromatic diamine represented by the following formula (1):

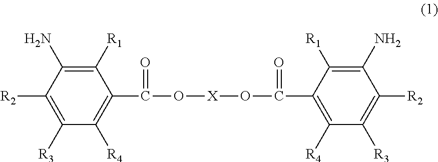

In the formula (1), X is selected from the following structures (a), (b) and (c),

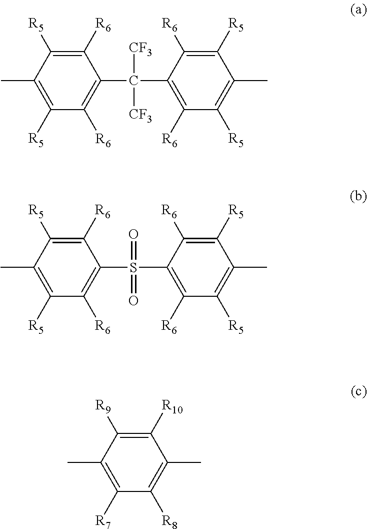

wherein R₁, R₂, R₃, and R₄ in the formula (1) and R₅, R₆, R₇, R₈, R₉ and R₁₀ in (a), (b) and (c) are, independently of each other, a hydrogen atom, an optionally substituted alkyl group having 1 to 6 carbon atoms, or an alkoxy group having 1 to 3 carbon atoms, provided that at least one of R₇, R₈, R₉ and R₁₀ is the alkyl group or the alkoxy group.

Another aspect of the present invention relates to a meta-type ester-containing aromatic diamine represented by the following formula (1′):

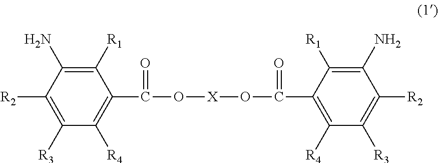

In the formula (1′), X is the following structure (d),

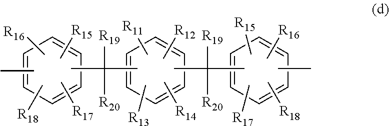

wherein R₁, R₂, R₃, R₄, R₁₁, R₁₂, R₁₃, R₁₄, R₁₅, R₁₆, R₁₇, R₁₈, R₁₉ and R₂₀ are, independently of each other, a hydrogen atom, an optionally substituted alkyl group having 1 to 6 carbon atoms, or an alkoxy group having 1 to 3 carbon atoms.

Examples of the optionally substituted alkyl group with 1 to 6 carbon atoms represented by R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉ and R₂₀ include methyl, ethyl, propyl, isopropyl, butyl, isobutyl, pentyl, neopentyl, cyclopentyl, hexyl, and cyclohexyl groups. Examples of the alkoxy group with 1 to 3 carbon atoms include methoxy, ethoxy, and propoxy groups. R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, and R₂₀ may be mutually different or may be the same. They are preferably the hydrogen atom or the alkyl group with 1 to 6 carbon atoms. More preferably, in the above-described structures (a), (b), and (d), all of R₁, R₂, R₃, R₄, R₅, R₆, R₁₁, R₁₂, R₁₃, R₁₄, R₁₅, R₁₆, R₁₇, and R₁₈ are the hydrogen atom. In the above-described (c), R₁, R₂, R₃, and R₄ are preferred to be the hydrogen atom and at least one of R₇, R₈, R₉, and R₁₀ is preferred to be the methyl group.

Preferably, the present compound is a tetranuclear compound represented by the following formula (1a) or (1b), or a trinuclear compound represented by the following formula (1c):

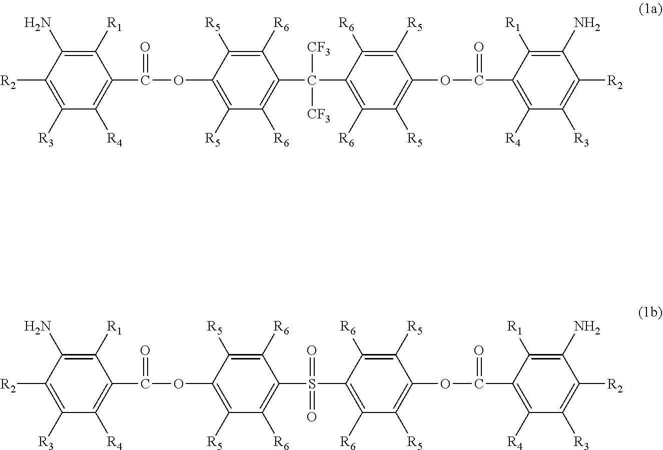

In the formulas (1a) and (1b), R₁, R₂, R₃, R₄, R₅ and R₆ are as described above, and are preferably the hydrogen atom.

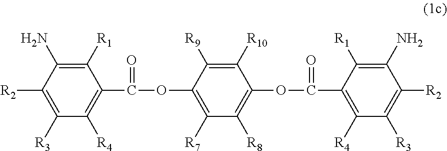

In the formula (1c), R₁, R₂, R₃ and R₄ are as described above and are preferably the hydrogen atom, and R₇, R₈, R₉, and R₁₀ are as described above and at least one of R₇, R₈, R₉, and R₁₀ is the methyl group.

The formula (d) is preferably represented by the following formula (1d).

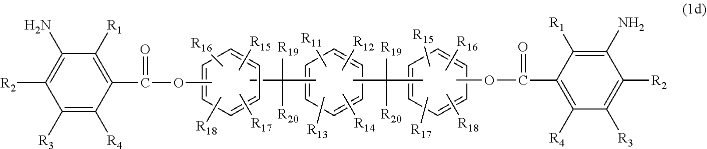

In the formula (1d), R₁, R₂, R₃, R₄, R₁₁, R₁₂, R₁₃, R₁₄, R₁₅, R₁₆, R₁₇ and R₁₈ are as described above and are preferably the hydrogen atom. R₁₉ and R₂₀ are as described above and are preferably the methyl group.

In the above-described formula (d), linkage positions of the substituents and the aromatic rings are not specifically limited. Preferred is a compound whose X has the following structure.

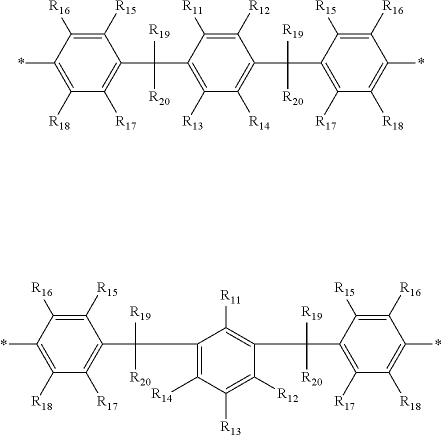

R₁, R₂, R₃, R₄, R₁₁, R₁₂, R₁₃, R₁₄, R₁₅, R₁₆, R₁₇, and R₁₈ are as described above and are preferably the hydrogen atom. R₁₉ and R₂₀ are as described above and are preferably the methyl groups. The sites marked with * in the formulas bond to the oxygen atom.

The compound of the present invention is further preferably to be the following compounds:

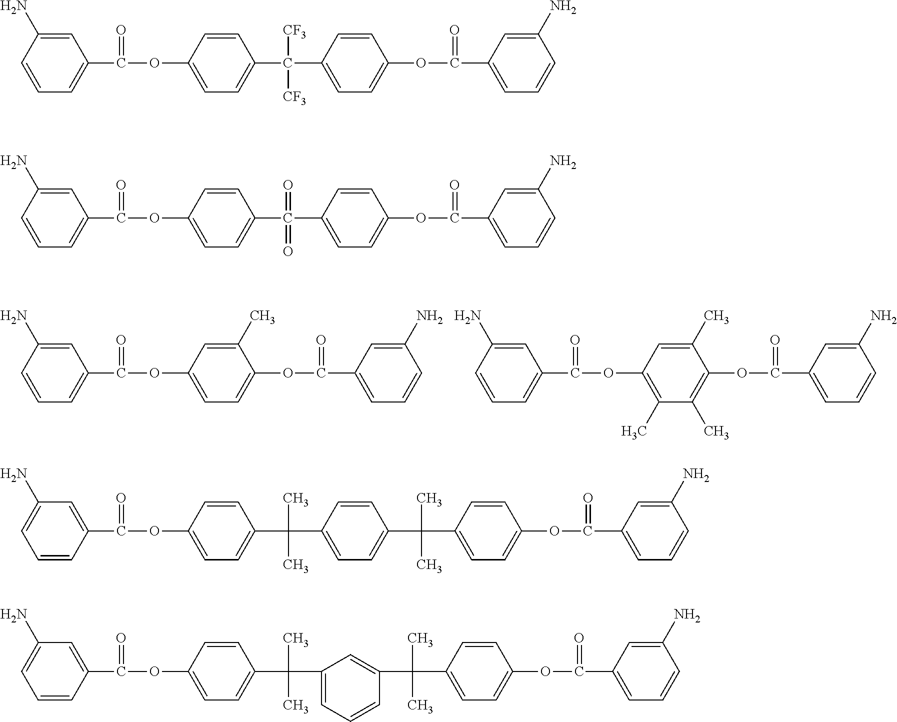

The compound represented by the above-described formula (1) is easily obtained by subjecting two nitro groups of the compound represented by the following formula (3) to reduction reaction,

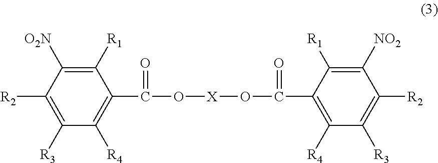

In the formula, R₁, R₂, R₃, R₄, and X are as described above.

The following describes the preparing method in more detail.

The reduction reaction of the above-described nitro groups is not specifically limited, and a known method that reduces a nitro group to an amino group may be used. Examples of a reduction method of an aromatic dinitro compound include catalytic reduction, Béchamp reduction, zinc dust reduction, tin chloride reduction, hydrazine reduction, and the like.

Examples of a solvent used in the reduction reaction include alcohol-based solvents such as methanol, ethanol, 1-propanol, isopropanol, 1-butanol, 2-methoxyethanol, and 2-ethoxyethanol; amide-based solvents such as N,N-dimethylformamide, N,N-dimethylacetamide, N-methylpyrrolidone, and N,N′-dimethylimidazolidinone; and ether-based solvents such as tetrahydrofuran, dioxane, ethylene glycol dimethyl ether, and diethylene glycol, but it is not limited to those as long as the aromatic dinitro compound dissolves in the solvent. The amount of the solvent is only necessary to be adjusted as necessary.

For the catalyst used in the reduction reaction, it may use a catalyst known as a catalyst for each reduction reaction described above. Examples of a catalyst used for the catalytic reduction or the hydrazine reduction include noble metal catalysts, such as palladium, platinum, and rhodium supported by activated carbon, carbon black, graphite, alumina, or the like, a Raney nickel catalyst, and a sponge nickel catalyst. The amount of the catalyst is not specifically limited, but is usually 0.1 to 10 wt % to the aromatic dinitro compound.

The reaction temperature and period of the reduction reaction may be selected as necessary. For example, the reaction may take place at a temperature in a range of 50 to 150° C., preferably at a temperature in a range of 60 to 130° C. for 1 to 35 hours, preferably 3 to 10 hours. The treatment method of the reaction product is not specifically limited. For example, after removing the catalyst and performing cooling, the generated solid matter is filtered, washed with water, and dried and, thus, the compound shown in the above-described general formula (1) is obtained. Furthermore, as necessary, if the compound is purified by a method, such as crystallization filtration and column separation, again, a highly purified product is obtained.

The compound represented by the above-described formula (3) is particularly preferably represented by the following formulas:

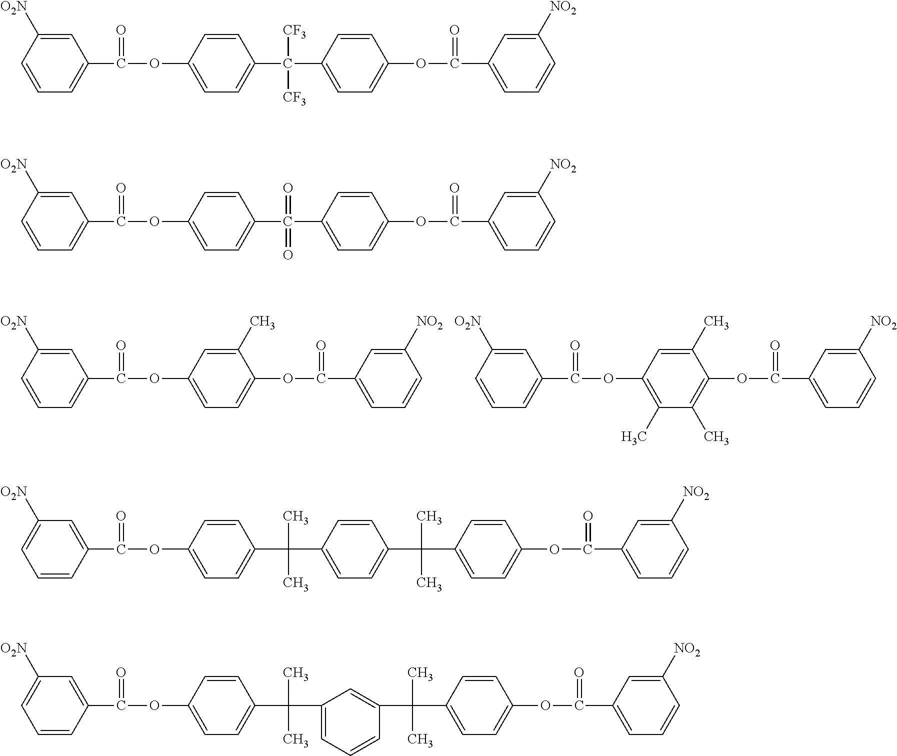

The compound represented by the above-described formula (3) may be prepared in a known method. For example, the compound may be prepared by a condensation of a diol compound and m-nitrobenzoic acid chloride corresponding to the respective compounds .

The meta-type ester-containing aromatic diamine represented by the above-described formula (1) is excellent in solubility in the various kinds of solvents and is effective as a raw material of polyimide. For example, the meta-type ester-containing aromatic diamine represented by the above-described formula (1) is reacted with an acid anhydride and thereby a polyimide compound is obtained.

The acid anhydride may be a conventionally-known one used as a raw material of polyimide. For example, it is at least one of acid dianhydrides selected from the group consisting of pyromellitic dianhydride, 3,3′,4,4′-biphenyltetracarboxylic dianhydride, 2,3,3′,4′-biphenyltetracarboxylic dianhydride, benzophenone-3,4,3′,4′-tetracarboxylic dianhydride, 4,4′-(2,2-hexafluoroisopropylidene) diphthalic dianhydride, 2,2-bis [3-(3,4-dicarboxyphenoxy) phenyl] propane dianhydride, 2,2-bis [4-(3,4-dicarboxyphenoxy) phenyl] propane dianhydride, and 3,3′,4,4′-diphenylsulfonetetracarboxylicdianhydride, and oxy-4,4′-diphthalic dianhydride.

The reactive condition and the reaction ratio between the diamine compound and the acid anhydride described above are not specifically limited, and may be selected as necessary according to a conventionally-known method. For example, for the reactive condition, the reaction may be performed at a temperature in a range of 25 to 30° C. for 0.5 to 24 hours. The reaction ratio may be 1.00. The obtained polyimide compound may preferably have a number average molecular weight of 2,000 to 200,000, and preferably 10,000 to 50,000. The number average molecular weight is a value measured by, for example, GPC (gel permeation chromatography, THF).

For the above-described polyimide compound, any diamine compound other than the diamine compounds of the present invention may be further reacted. The proportion of a unit derived from the diamine compound of the present invention is preferred to be 10 mol % to 100 mol %, relative to the total molar quantity of units derived from all the diamine compounds in the polyimide compound. Any diamine compound other than the diamine compounds of the present invention is, for example, one or more selected from the group consisting of 1,4-phenylenediamine, 1,3-phenylenediamine, 1,2-phenylenediamine, 2,4-diaminotoluene, 2,6-diaminotoluene, m-xylylenediamine, p-xylylenediamine, 2,2′-dimethylbenzidine, 3,3′-dimethylbenzidine, 2,2′-bis(trifluoromethyl)benzidine, 4,4′-diaminodiphenyl ether, 3,4′-diaminodiphenyl ether, 3,3′-diaminodiphenyl ether, 4,4′-diaminodiphenyl sulfone, 3,3′-diaminodiphenyl sulfone, 4,4′-diaminodiphenyl sulfide, 4,4′-diaminobenzanilide, 1,3-bis(4-aminophenoxy)benzene, 1,4-bis(4-aminophenoxy)benzene, 1,3-bis(3-aminophenoxy)benzene, 4,4′-bis (4-aminophenoxy)biphenyl, bis[4-(3-aminophenoxy)phenyl] sulfone, bis[4-(4-aminophenoxy)phenyl]sulfone, 2,2-bis[4-(4-aminophenoxy)phenyl]propane, and 9,9′-bis(4-aminophenyl)fluorene, and 9,9′-bis[4-(4-aminophenoxy)phenyl]fluorene.

Examples of molded products comprising the polyimide compound of the present invention include a material for high speed and large capacity communication.

### EXAMPLES

The present invention will be explained below in further detail with reference to a series of the Examples, though the present invention is in no way limited by these Examples.

The measuring method and device used in the following Examples are follows.

HPLC measurement was conducted by SPD-20A produced by SHIMADZU and melting point measurement was conducted by MP-21 produced by YAMATO.

¹H nuclear magnetic resonance spectrum analysis was conducted by Avance iii HD 400 (Bruker Biospin) with deuterated DMSO used as a measurement solvent.

¹³C nuclear magnetic resonance spectrum analysis was conducted by Avance iii HD 400 (Bruker Biospin) with deuterated DMSO used as a measurement solvent.

Infrared spectroscopy was conducted by FT/IR-4700 produced by JASCO Corporation according to an ATR method.

Accurate mass analysis was conducted by Xevo g2-XS QTof produced by Waters.

### Example 1

Synthesis of 2,2′-bis[4-(3-nitrobenzoyloxy)phenyl]hexafluoropropane

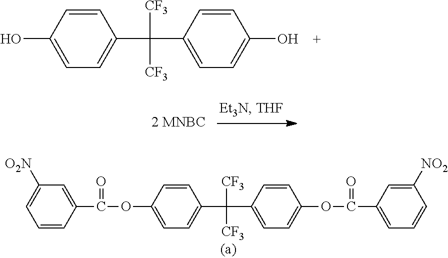 [SMILES: Oc1ccc(C(c2ccc(O)cc2)(C(F)(F)F)C(F)(F)F)cc1 ; O=C(Oc1ccc(C(c2ccc(OC(=O)c3cccc([N+](=O)[O-])c3)cc2)(C(F)(F)F)C(F)(F)F)cc1)c1cccc([N+](=O)[O-])c1]

In a 300 mL four-necked flask equipped with a mechanical stirrer and a thermometer, were placed 25.2 g (75 mmol) of bisphenol AF, 200 mL of THE (tetrahydrofuran), and 16.0 g (158 mmol) of triethylamine, and were dissolved at room temperature to obtain a light-yellow transparent solution. To the solution, 25.0 g (158 mmol) of MNBC (m-nitrobenzoyl chloride) was added and, then, a white precipitation was immediately generated. The internal temperature increased from 25° C. to 55° C., and was cooled down after a short time. The mixture was stirred as it is at room temperature for one hour, and the dissipation of the MNBC was confirmed by the HPLC. While the temperature was kept at room temperature, triethylamine hydrochloride was filtered out and the solvent was distilled away using an evaporator to obtain white solid. The white solid matter was slurry washed with 300 mL of ion exchanged water and filtered, and the obtained cake was thermally dissolved in 260 mL of acetonitrile. The mixture was slowly cooled down to 5° C., was filtered and dried and, thereby, to obtain a product with white needle crystal of 36.0 g/yield of 76%, mp of 195.2 to 196.5° C., and HPLC purity of 98.7%. The product was 2,2′-bis[4-(3-nitrobenzoyloxy)phenyl]hexafluoropropane represented by the above-described formula (a), hereinafter referred to as a dinitro compound 1.

TOF-MS (ESI): 633.073 (M)⁻

### Example 2

Synthesis of 2,2′-bis[4-(3-aminobenzoyloxy)phenyl]hexafluoropropane

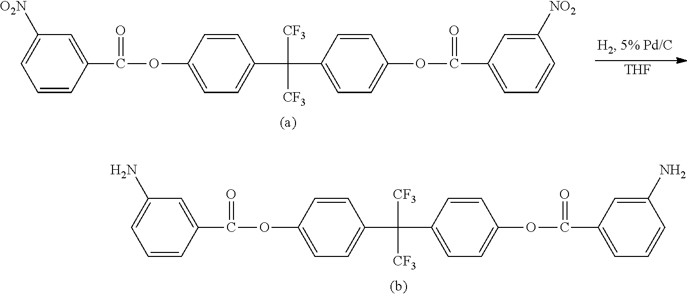 [SMILES: O=C(Oc1ccc(C(c2ccc(OC(=O)c3cccc([N+](=O)[O-])c3)cc2)(C(F)(F)F)C(F)(F)F)cc1)c1cccc([N+](=O)[O-])c1 ; Nc1cccc(C(=O)Oc2ccc(C(c3ccc(OC(=O)c4cccc(N)c4)cc3)(C(F)(F)F)C(F)(F)F)cc2)c1 ; "THF"]

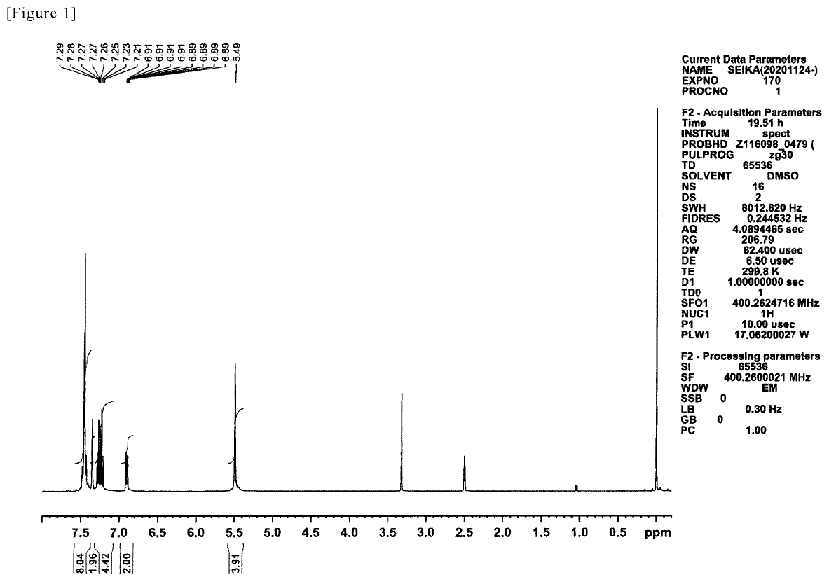

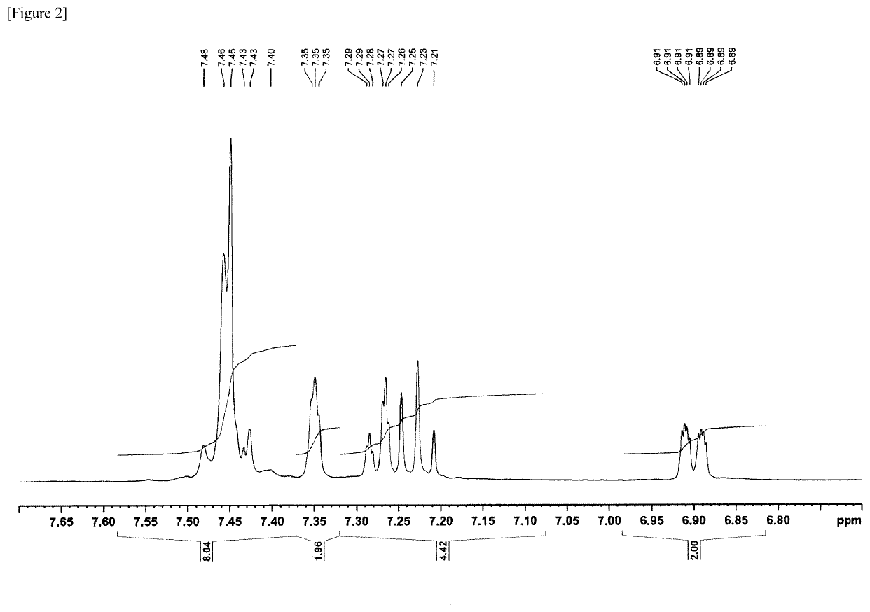

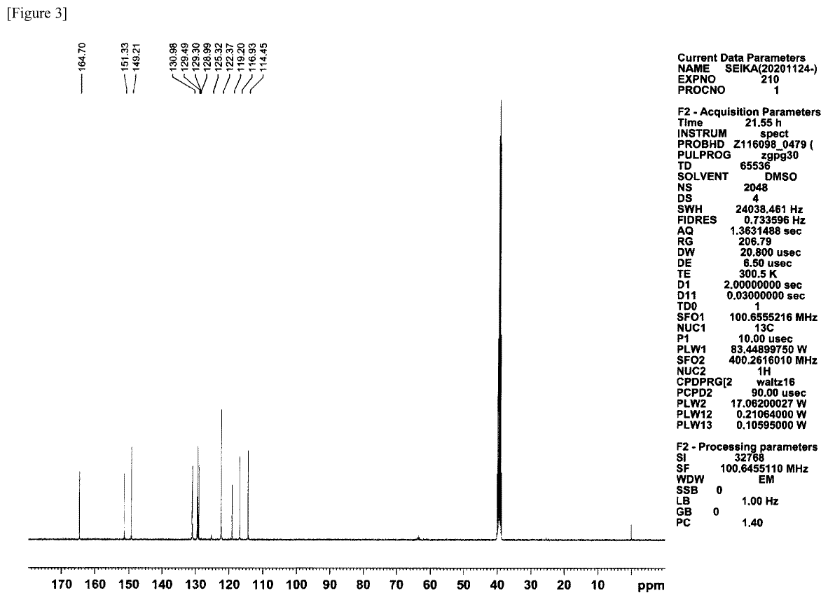

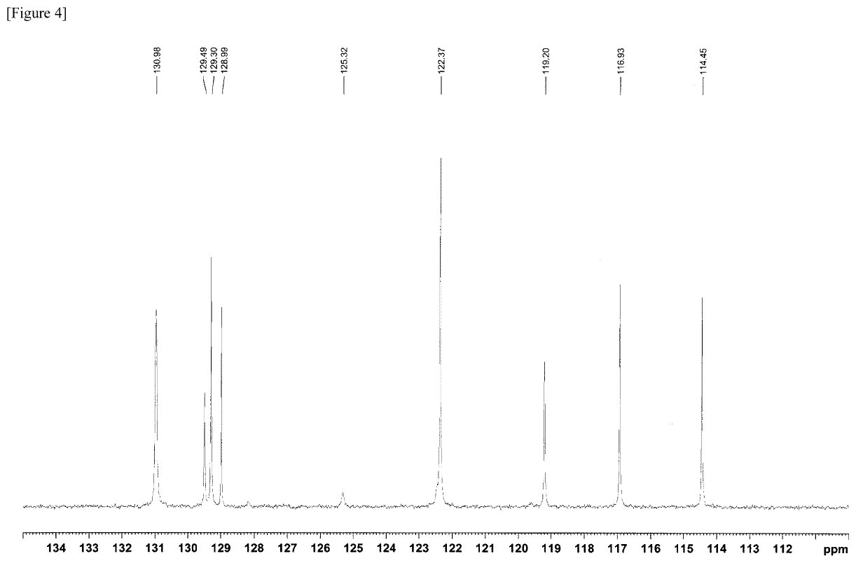

In a 300 mL SUS autoclave, were placed 22.5 g (35 mmol/purity conversion) of the dinitro compound 1 obtained in the above-described Example 1, 0.261 g (0.113 g as Dry) of 5% Pd/C, and 150 mL of THE and were sealed. Four times of nitrogen substitution and four times of hydrogen substitution were repeated, and no gas leakage was confirmed with a soap solution. The temperature of the mixture was increased to 50° C. at 150 rpm stirring under hydrogen 0.8 MPa constant pressure. The frequency of stirring was increased to 1000 rpm, and a hydrogen introduction valve was opened. While the internal temperature was kept at 60 to 65° C., a theoretical quantity of hydrogen was absorbed in 38 minutes, was aged for another 10 minutes, and it was confirmed that the internal pressure did not drop. After the nitrogen substitution, the autoclave was opened, and the used catalyst was hot-filtered. The solvent was distilled away from the hydrogenation reaction mother liquid using an evaporator, and the obtained white solid matter was thermally dissolved in 150 mL of isopropanol, to which 0.4 g of activated carbon was added, and was stirred for 30 minutes under reflux. The activated carbon was filtered out and 90 mL of ion exchanged water was added. The generated precipitation was thermally dissolved, was slowly cooled down to 5° C., was filtered and dried and thereby to obtain a product with pale yellow needle crystal of 18.5 g/yield of 92%, mp of 159.6 to 160.5° C., and HPLC purity of 99.6%. The product was structurally analyzed by ¹H-NMR and ¹³C-NMR. The results are shown in FIG. 1 to FIG. 4. The product was 2,2′-bis[4-(3-aminobenzoyloxy)phenyl]hexafluoropropane represented by the above-described formula (b).

TOF-MS (ESI): 575.1414 (M+H)⁺

### Example 3

Synthesis of bis[4-(3-nitrobenzoyloxy)phenyl]sulfone

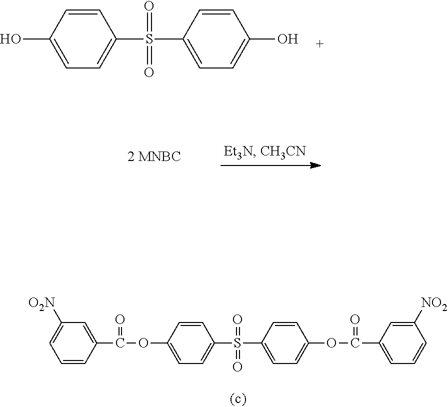 [SMILES: O=S(=O)(c1ccc(O)cc1)c1ccc(O)cc1 ; O=C(Oc1ccc(S(=O)(=O)c2ccc(OC(=O)c3cccc([N+](=O)[O-])c3)cc2)cc1)c1cccc([N+](=O)[O-])c1]

In a 300 mL four-necked flask equipped with a mechanical stirrer and a thermometer, were placed 12.8 g (51 mmol) of bisphenol S, 200 mL of acetonitrile, 16.0 g (158 mmol) of triethylamine, and their temperature was increased to 50° C. to obtain white slurry. To the slurry, 25.0 g (158 mmol) of MNBC (m-nitrobenzoyl chloride) was added and, then, the internal temperature increased from 50° C. to 70° C. and was cooled down after a short time. While the temperature was kept at 60° C., the mixture was stirred for one hour. A white precipitation was filtered out at 50° C., and the cake was washed with methanol. The cake was air-dried and, thereby, to obtain a product with a white powder of 27.3 g/yield of 95%, mp of 252° C. to 253° C., and HPLC purity of 98%. The product was bis[4-(3-nitrobenzoyloxy)phenyl]sulfone represented by the above-described formula (c), hereinafter referred to as a dinitro compound 2.

### Example 4

Synthesis of bis[4-(3-aminobenzoyloxy)phenyl]sulfone

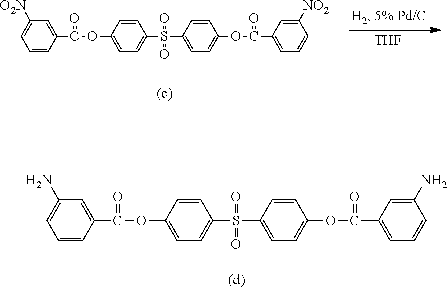 [SMILES: O=C(Oc1ccc(S(=O)(=O)c2ccc(OC(=O)c3cccc([N+](=O)[O-])c3)cc2)cc1)c1cccc([N+](=O)[O-])c1 ; Nc1cccc(C(=O)Oc2ccc(S(=O)(=O)c3ccc(OC(=O)c4cccc(N)c4)cc3)cc2)c1 ; "THF"]

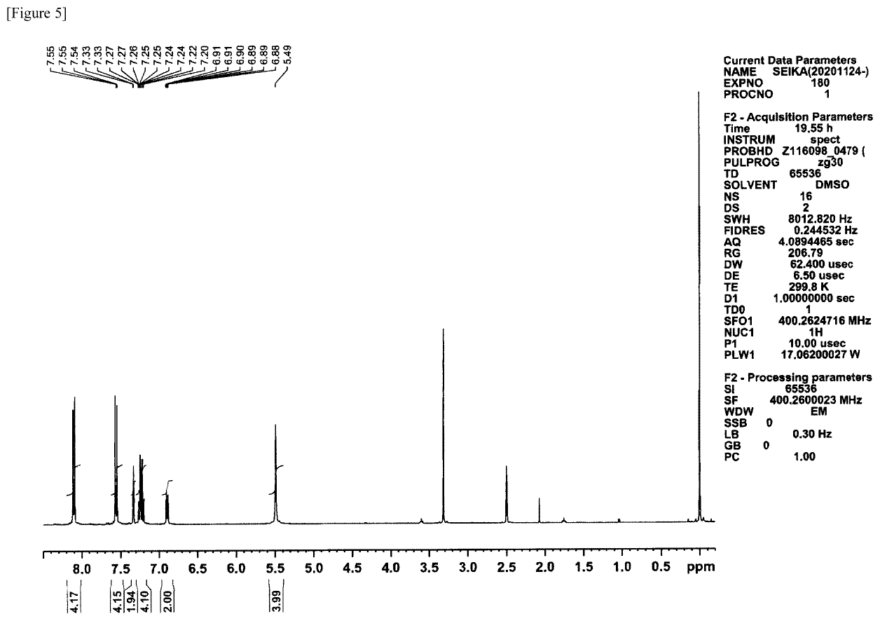

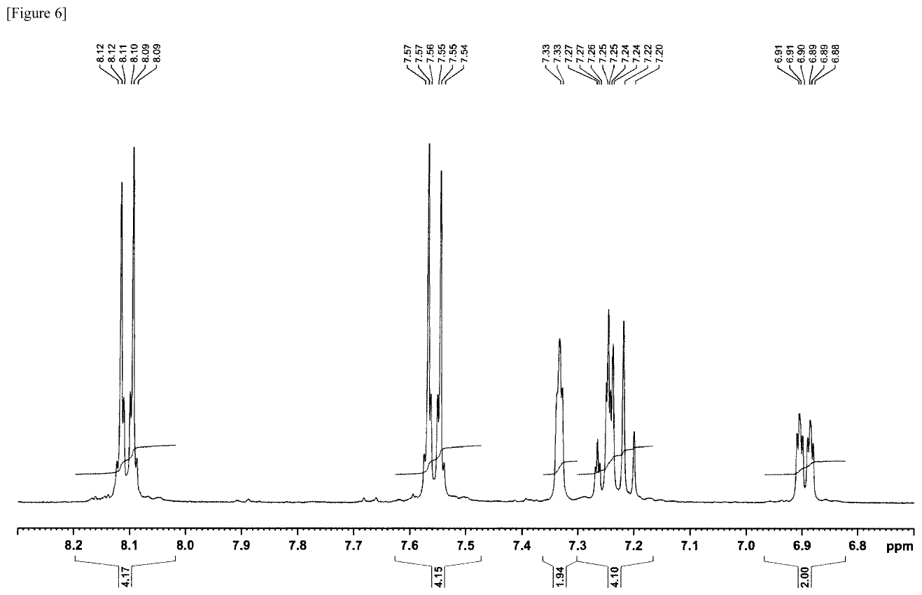

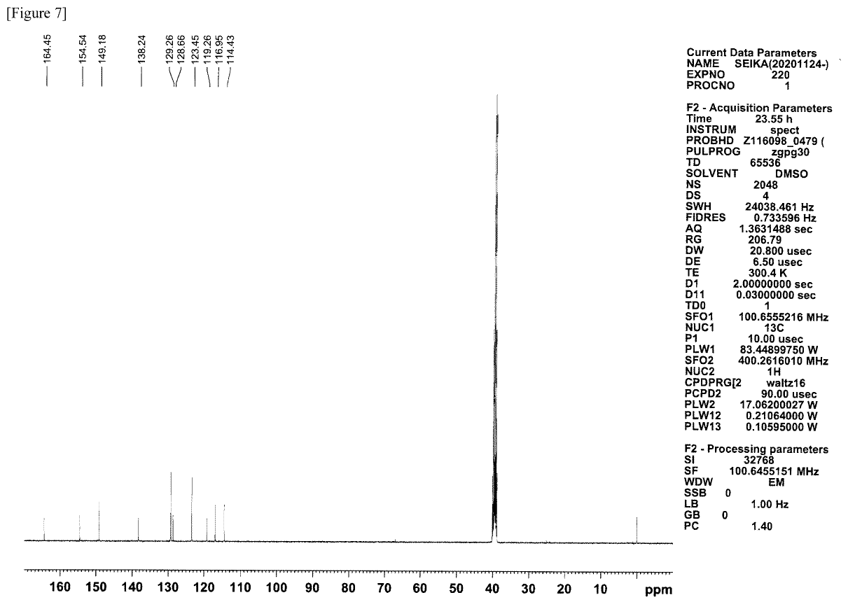

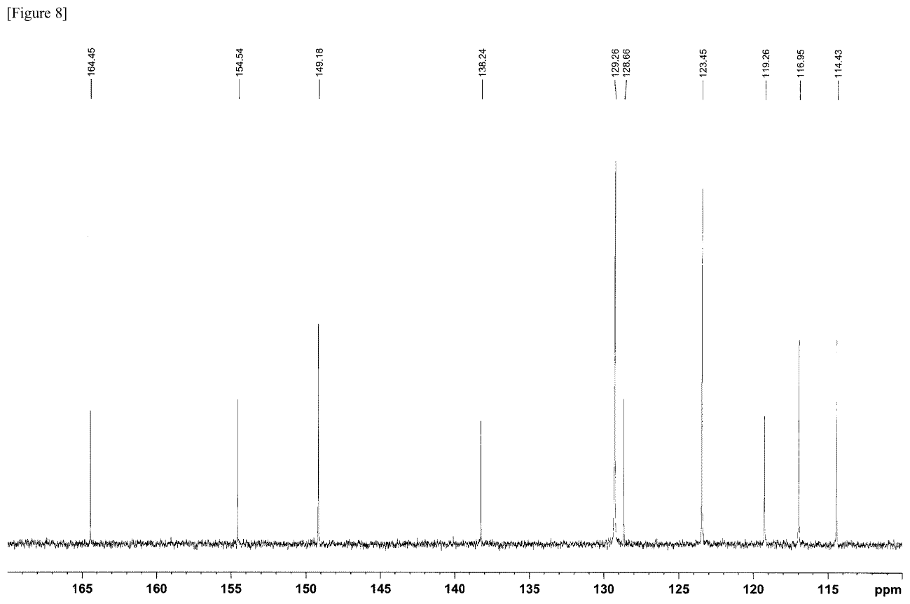

In a 300 mL SUS autoclave, were placed 22.5 g (35 mmol/purity conversion) of the dinitro compound 2 obtained in the above-described Example 3, 0.261 g (0.113 g as Dry) of 5% Pd/C, and 150 mL of THE and were sealed. Four times of nitrogen substitution and four times of hydrogen substitution were repeated, and no gas leakage was confirmed with a soap solution. The temperature of the mixture was increased to 50° C. at 150 rpm stirring under hydrogen 0.8 MPa constant pressure. The frequency of stirring was increased to 1000 rpm, and a hydrogen introduction valve was opened. While the internal temperature was kept at 60 to 65° C., a theoretical quantity of hydrogen was absorbed in 85 minutes, was aged for another 20 minutes, and it was confirmed that the internal pressure did not drop. After the nitrogen substitution, the autoclave was opened resulting in finding diamine deposition and, therefore, the solvent was directly distilled away from the hydrogenation reaction mother liquid using an evaporator, and the mixture was thermally dissolved in 350 mL of acetonitrile, to which 0.4 g of activated carbon was added, and was stirred for 30 minutes under reflux. The activated carbon was filtered out and 40 mL of ion exchanged water was added. The generated precipitation was thermally dissolved, was slowly cooled down to 5° C., was filtered and dried and, thereby, to obtain a product with a pale-yellow crystalline powder of 14.5 g/yield of 74%, mp of 235 to 236° C., and HPLC purity of 94%. The product was structurally analyzed by ¹H-NMR and ¹³C-NMR. The results are shown in FIG. 5 to FIG. 8. The product was bis[4-(3-aminobenzoyloxy)phenyl]sulfone represented by the above-described formula (d).

TOF-MS (ESI): 489.1106 (M+H)⁺

### Example 5

Synthesis of 1-methyl-2,5-bis(3-nitrobenzoyloxy)benzene

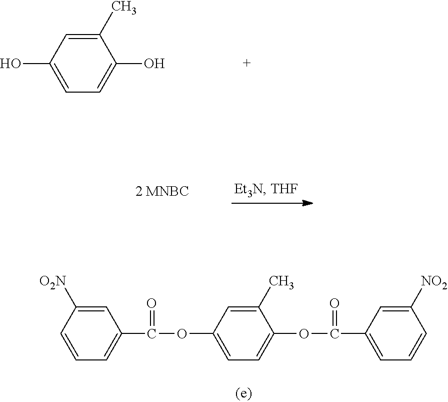

In a 300 mL four-necked flask equipped with a mechanical stirrer and a thermometer, were placed 9.3 g (75 mmol) of methylhydroquinone, 200 mL of THE, and 16.0 g (158 mmol) of triethylamine and were dissolved at room temperature to obtain a colorless transparent solution. To the solution, 25.0 g (158 mmol) of MNBC (m-nitrobenzoyl chloride) was added and, then, a white precipitation was immediately generated. The internal temperature increased from 25° C. to 58° C., and was cooled down after a short time. The mixture was stirred as it is at room temperature for one hour, and the dissipation of the MNBC was confirmed by the HPLC. While the temperature was kept at room temperature, the white precipitation was filtered out, the cake was washed with THE, and the slurry was washed with 400 mL of ion exchanged water for 30 minutes at 60° C. While the temperature was kept at 60° C., the cake was filtered and washed with methanol. The cake was air-dried and, thereby, to obtain a product with a white powder of 24.6 g/yield of 78%, mp of 198.0° C. to 199.2° C., and HPLC purity of 99.3%. The product was 1-methyl-2,5-bis(3-nitrobenzoyloxy)benzene represented by the above-described formula (e), hereinafter referred to as a dinitro compound 3.

### Example 6

Synthesis of 1-methyl-2,5-bis(3-aminobenzoyloxy)benzene

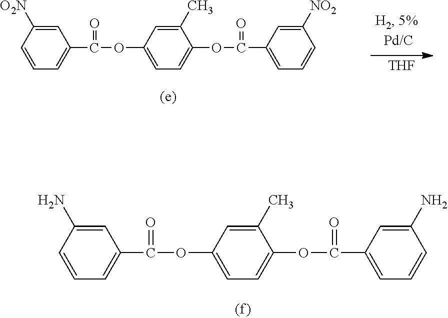 [SMILES: Cc1cc(OC(=O)c2cccc([N+](=O)[O-])c2)ccc1OC(=O)c1cccc([N+](=O)[O-])c1 ; Cc1cc(OC(=O)c2cccc(N)c2)ccc1OC(=O)c1cccc(N)c1 ; "THF"]

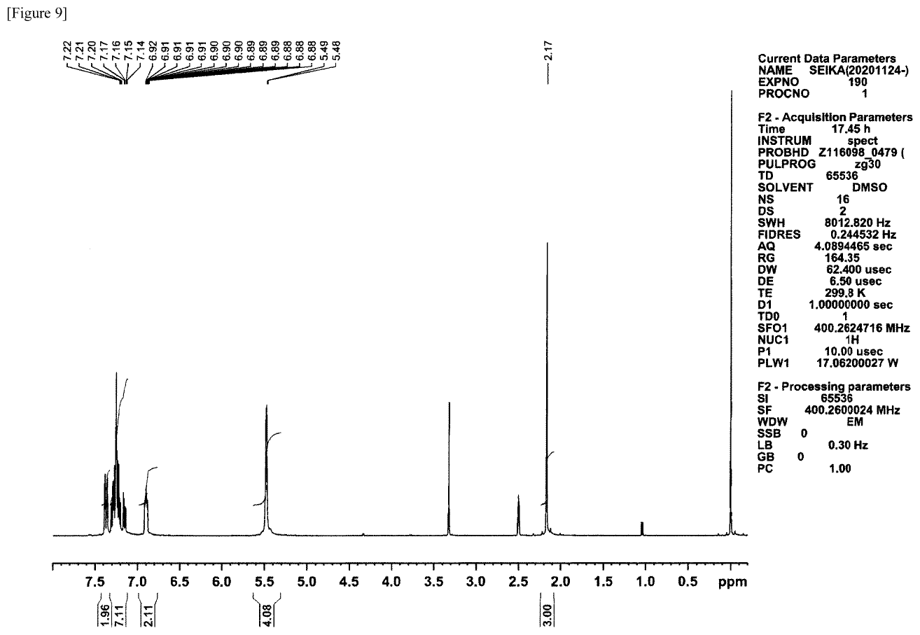

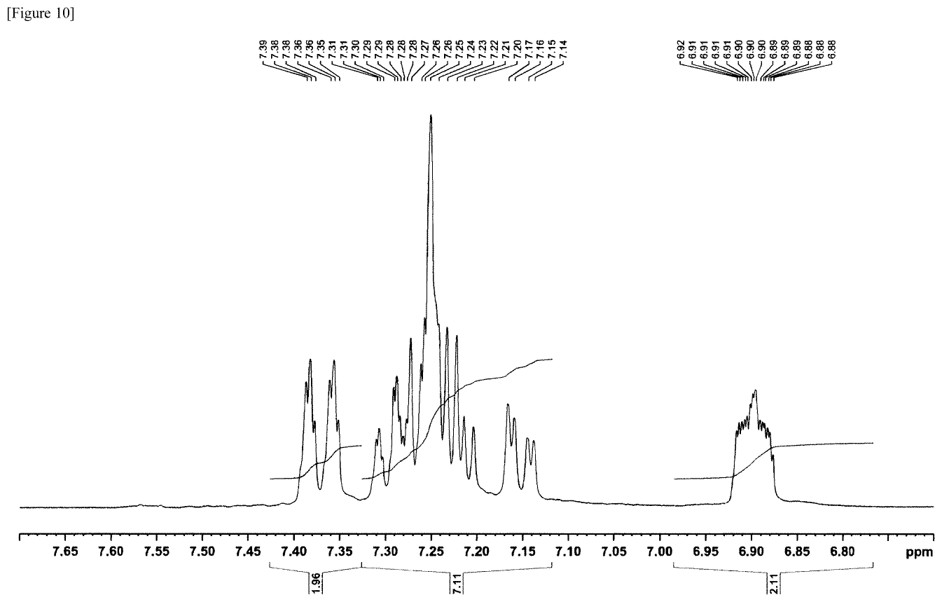

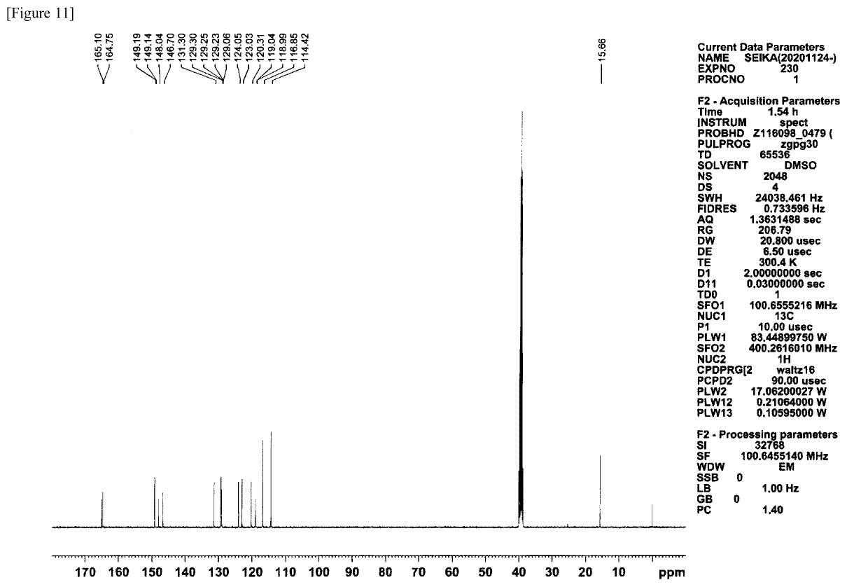

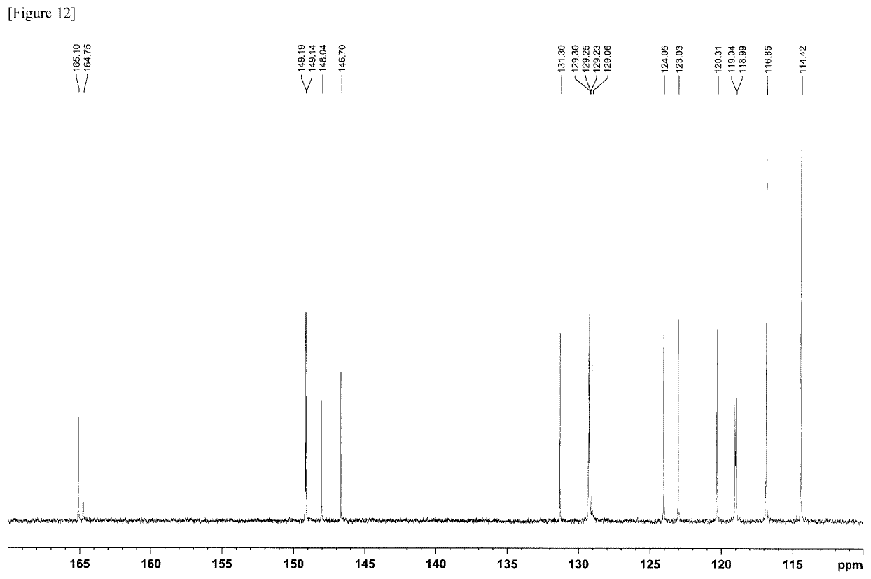

In a 300 mL SUS autoclave, were placed 22.5 g (35 mmol/purity conversion) of the dinitro compound 3 obtained in the above-described Example 5, 0.261 g (0.113 g as Dry) of 5% Pd/C, and 150 mL of THE and were sealed. Four times of nitrogen substitution and four times of hydrogen substitution were repeated, and no gas leakage was confirmed with a soap solution. The temperature of the mixture was increased to 50° C. at 150 rpm stirring under hydrogen 0.8 MPa constant pressure. The frequency of stirring was increased to 1000 rpm, and a hydrogen introduction valve was opened. While the internal temperature was kept at 60 to 65° C., a theoretical quantity of hydrogen was absorbed in 30 minutes, was aged for another 10 minutes, and it was confirmed that the internal pressure did not drop. After the nitrogen substitution, the autoclave was opened, and the used catalyst was hot-filtered. The solvent was distilled away from the hydrogenation reaction mother liquid using an evaporator, and the obtained white solid matter was thermally dissolved in 500 mL of isopropanol, to which 0.4 g of activated carbon was added, and was stirred for 30 minutes under reflux. The activated carbon was filtered out and 500 mL of ion exchanged water was added. The generated precipitation was thermally dissolved, was slowly cooled down to 5° C., was filtered and dried and, thereby, to obtain a product with a pale-yellow crystalline powder of 11.7 g/yield of 61%, mp of 148 to 150° C., and HPLC purity of 96%. The product was structurally analyzed by ¹H-NMR and ¹³C-NMR. The results are shown in FIG. 9 to FIG. 12. The product was 1-methyl-2,5-bis(3-aminobenzoyloxy)benzene represented by the above-described formula (f).

TOF-MS (ESI): 363.1336 (M+H)⁺

### Example 7

Synthesis of 1,2,4-trimethyl-3,6-bis(3-nitrobenzoyloxy)benzene

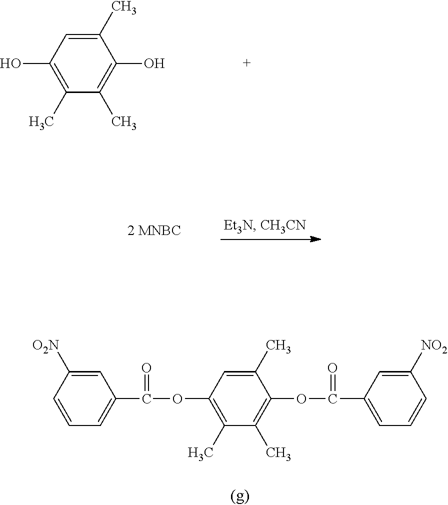 [SMILES: Cc1cc(O)c(C)c(C)c1O ; Cc1cc(OC(=O)c2cccc([N+](=O)[O-])c2)c(C)c(C)c1OC(=O)c1cccc([N+](=O)[O-])c1]

In a 300 mL four-necked flask equipped with a mechanical stirrer and a thermometer, were placed 11.4 g (75 mmol) of trimethylhydroquinone, 200 mL of acetonitrile, and 16.0 g (158 mmol) of triethylamine and were mixed and solved at room temperature to obtain a colorless transparent solution. To the solution, 25.0 g (158 mmol) of MNBC (m-nitrobenzoyl chloride) was added, immediately, a pale-yellow precipitation was generated. The internal temperature increased from 18° C. to 57° C., and the viscosity also increased. The viscosity gradually reduced by heating to 70° C., after two hours, the temperature was cooled down to 25° C., and a white precipitation was filtered out and the cake was washed with methanol. The cake was air-dried and, thereby, to obtain a product with a white powder of 27.3 g/yield of 81%, mp of 226.0° C. to 226.8° C., and HPLC purity of 99.9%. The product was 1,2,4-trimethyl-3,6-bis(3-nitrobenzoyloxy)benzene represented by the above-described formula (g), hereinafter referred to as a dinitro compound 4.

### Example 8

Synthesis of 1,2,4-trimethyl-3,6-bis(3-aminobenzoyloxy)benzene

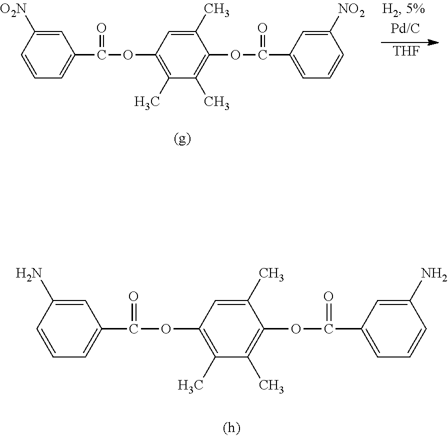 [SMILES: Cc1cc(OC(=O)c2cccc([N+](=O)[O-])c2)c(C)c(C)c1OC(=O)c1cccc([N+](=O)[O-])c1 ; Cc1cc(OC(=O)c2cccc(N)c2)c(C)c(C)c1OC(=O)c1cccc(N)c1 ; "THF"]

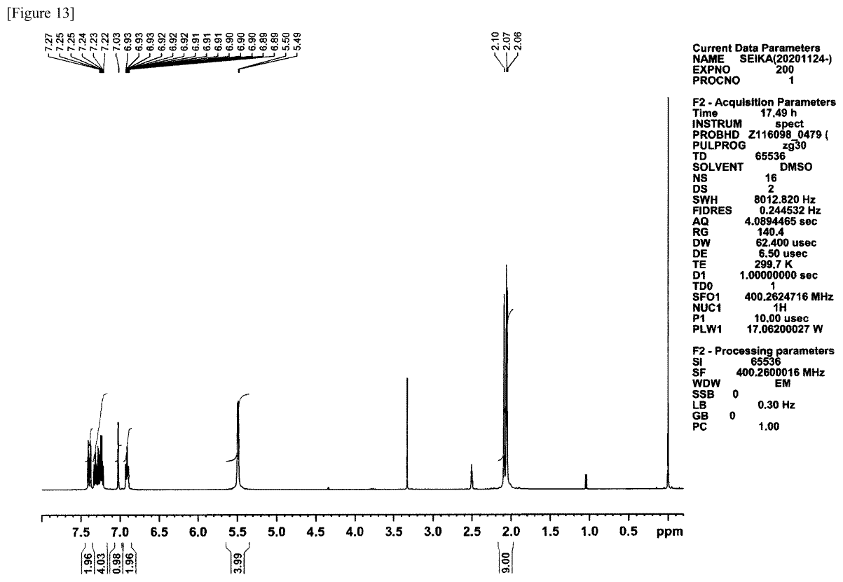

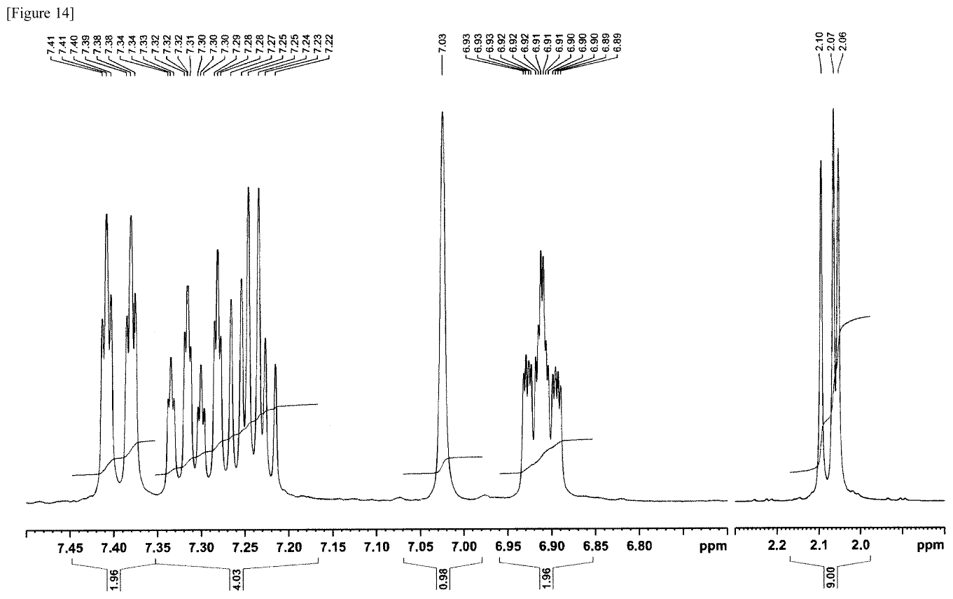

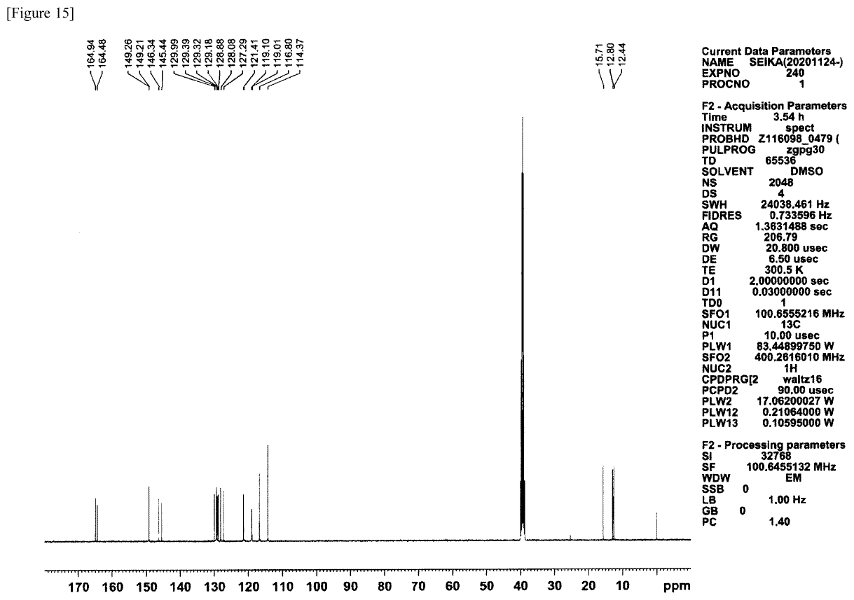

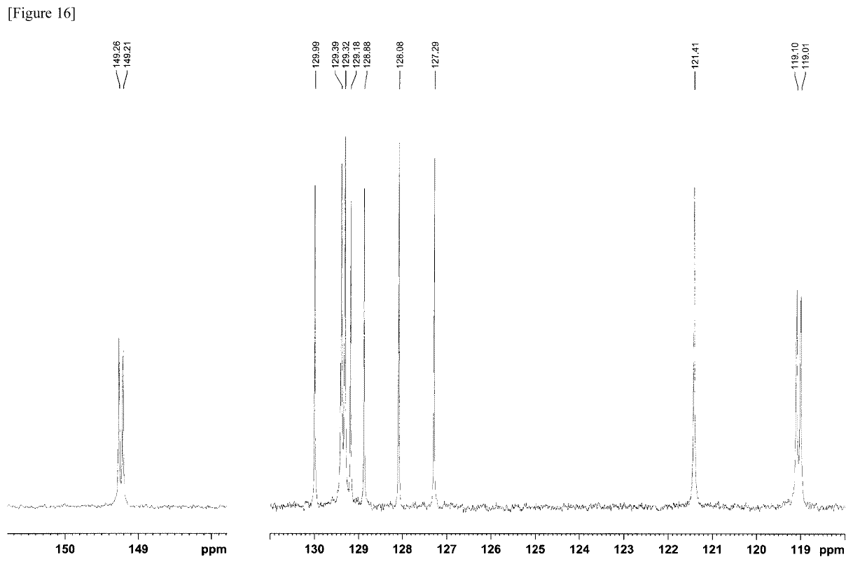

In a 300 mL SUS autoclave, were placed 22.5 g (35 mmol/purity conversion) of the dinitro compound 4 obtained in the above-described Example 7, 0.261 g (0.113 g as Dry) of 5% Pd/C, and 150 mL of THE and were sealed. Four times of nitrogen substitution and four times of hydrogen substitution were repeated, and no gas leakage was confirmed with a soap solution. The temperature of the mixture was increased to 50° C. at 150 rpm stirring under hydrogen 0.8 MPa constant pressure. The frequency of stirring was increased to 1000 rpm, and a hydrogen introduction valve was opened. While the internal temperature was kept at 60 to 65° C., a theoretical quantity of hydrogen was absorbed in 50 minutes, was aged for another 10 minutes, and it was confirmed that the internal pressure did not drop. After the nitrogen substitution, the autoclave was opened, and the used catalyst was hot-filtered. The solvent was distilled away from the hydrogenation reaction mother liquid using an evaporator, the obtained white solid matter was thermally dissolved in 500 mL of isopropanol, to which 0.4 g of activated carbon was added, and was stirred for 30 minutes under reflux. The activated carbon was filtered out and 500 mL of ion exchanged water was added. The generated precipitation was thermally dissolved, was slowly cooled down to 5° C., and the primary crystal was filtered. The filtrate was condensed to ⅔ and the generated secondary crystal was filtered, which was dried together with the previous primary crystal and, thereby, to obtain a product with a pale-yellow crystalline powder of 18.9 g/yield of 94%, mp of 187 to 189° C., and HPLC purity of 97%. The product was structurally analyzed by ¹H-NMR and ¹³C-NMR. The results are shown in FIG. 13 to FIG. 16. The product was 1,2,4-trimethyl-3,6-bis(3-aminobenzoyloxy)benzene represented by the above-described formula (h).

TOF-MS (ESI): 391.1643 (M+H)⁺

### Example 9

Synthesis of [1,4-phenylenebis(propane-2,2-diyl)]bis(4,1-phenylene)bis(3-nitrobenzoate)

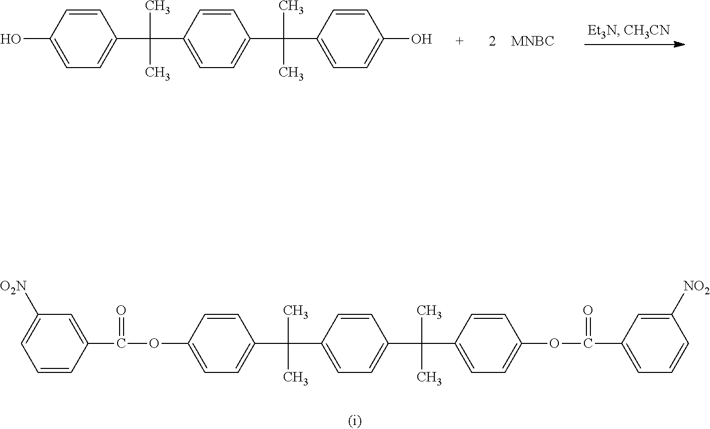 [SMILES: CC(C)(c1ccc(O)cc1)c1ccc(C(C)(C)c2ccc(O)cc2)cc1 ; CC(C)(c1ccc(OC(=O)c2cccc([N+](=O)[O-])c2)cc1)c1ccc(C(C)(C)c2ccc(OC(=O)c3cccc([N+](=O)[O-])c3)cc2)cc1]

In a 500 mL four-necked flask equipped with a stirrer, a thermometer, a Dean-Stark apparatus, and a Dimroth condenser, were placed 26.0 g (72 mmol) of bis(4-hydroxyphenyl)-1,4-diisopropylbenzene, 200 mL of acetonitrile, and 16.0 g (158 mmol) of triethylamine, and were stirred at 300 rpm to obtain white slurry. To the slurry, 25.0 g (158 mmol) of MNCB (m-nitrobenzoyl chloride) was added and stirred at 75° C. for three hours (the internal temperature increased to 50° C. as soon as the MNCB was added). The mixture was cooled down to 25° C., the white precipitation was filtered with Kiriyama funnel having 110 mmφ with a No. 5C filter paper, was immersed and washed with 100 mL of methanol, was immersed and washed with 200 mL of ion exchanged water, and was dried under reduced pressure at 90° C., −0.1 MPa for 16 h and, thereby, to obtain 40.6 g of nitro compound in a white powder with yield of 88%, LC purity (Area %) of 98.8%, and melting point (visual observation) of 208 to 209° C. The product was [1,4-phenylenebis(propane-2,2-diyl)]bis(4,1-phenylene)bis(3-nitrobenzoate) represented by the above-described formula (i), hereinafter referred to as a dinitro compound 5.

TOF-MS (ESI): 643.209 (M−H)⁻

### Example 10

Synthesis of [1,4-phenylenebis(propane-2,2-diyl)]bis(4,1-phenylene)bis(3-aminobenzoate)

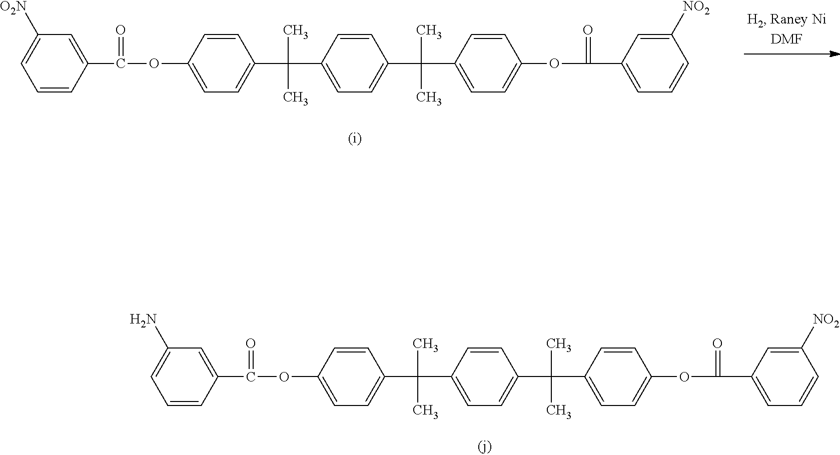 [SMILES: CC(C)(c1ccc(OC(=O)c2cccc([N+](=O)[O-])c2)cc1)c1ccc(C(C)(C)c2ccc(OC(=O)c3cccc([N+](=O)[O-])c3)cc2)cc1 ; CC(C)(c1ccc(OC(=O)c2cccc(N)c2)cc1)c1ccc(C(C)(C)c2ccc(OC(=O)c3cccc([N+](=O)[O-])c3)cc2)cc1]

In a 300 mL autoclave, were placed 15.0 g (23 mmol) of the dinitro compound 5, 100 mL of DMF, and 1.0 g of Raney-Ni and were sealed. Four times of nitrogen substitution and four times of hydrogen substitution were repeated, the internal pressure of the autoclave was prepared to be 0.8 MPa and a leakage was confirmed, and after no hydrogen leakage was confirmed, a hydrogen introduction valve was closed and sealed. While being stirred at 200 rpm, the mixture was heated with a preheated heating mantle, the stirring rate was set to 1000 rpm at the timing where the temperature reached 90° C., the hydrogen introduction valve was opened and, thus, a hydrogenation reaction was started (this timing was set to reaction start 0 min). The reaction was performed at 90±1° C., under 0.80 MPa constant pressure. The reaction was performed until the instantaneous hydrogen absorption was no longer observed with a large flowmeter. At this time, the hydrogenation period was 50 min. The hydrogen introduction valve was closed, the mixture was stirred for 60 min, no drop in the internal pressure was confirmed and the stirring was stopped, after the hydrogen in the autoclave was exhausted, the nitrogen substitution (gauge pressure of 0 to 0.3 MPa) was performed for three times. The diamine that has opened the autoclave was deposited and, therefore, the solvent was distilled away using an evaporator, 200 mL of acetonitrile was added to thermally dissolve the mixture, and the catalyst was filtered. The filtrate was cooled down to 5° C. and a pale grayish white powder was deposited at approximately 20° C. The powder was filtered with Kiriyama funnel having a 110 mmφ with a No. 5C filter paper, was immersed and washed with 30 mL of methanol and 30 mL of ion exchanged water, was dried under reduced pressure at 90° C., −0.1 MPa for 16 h and, thereby, to obtain 7.9 g of a pale grayish white powder with yield of 97%, LC purity of 98.6%, and melting point (visual observation) of 284 to 285° C. The product was [1,4-phenylenebis(propane-2,2-diyl)]bis(4,1-phenylene)bis(3-aminobenzoate) represented by the above-described formula (j).

TOF-MS (ESI): 585.276 (M+H)⁺

### Example 11

Synthesis of [1,3-phenylenebis(propane-2,2-diyl)]bis(4,1-phenylene)bis(3-nitrobenzoate)

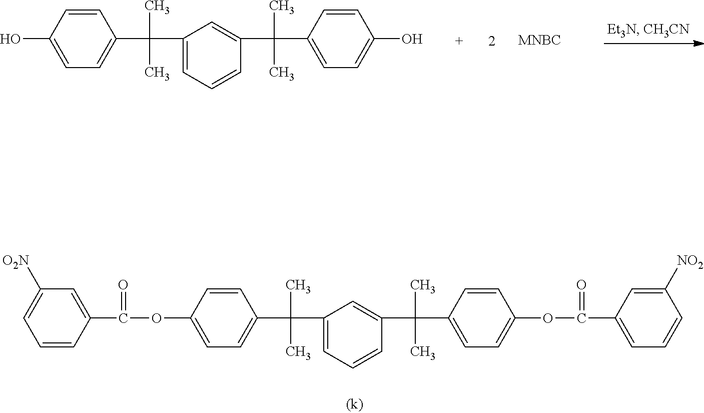 [SMILES: CC(C)(c1ccc(O)cc1)c1cccc(C(C)(C)c2ccc(O)cc2)c1 ; CC(C)(c1ccc(OC(=O)c2cccc([N+](=O)[O-])c2)cc1)c1cccc(C(C)(C)c2ccc(OC(=O)c3cccc([N+](=O)[O-])c3)cc2)c1]

In a 500 mL four-necked flask equipped with a stirrer, a thermometer, a Dean-Stark apparatus, and a Dimroth condenser, were placed 26.0 g (75 mmol) of bisphenol M, 200 mL of acetonitrile, and 16.0 g (158 mmol) of triethylamine, and were stirred at 300 rpm to obtain colorless transparent mixture. To the mixture, 25.0 g (158 mmol) of MNCB (m-nitrobenzoyl chloride) was added and stirred at 60° C. for two hours (the internal temperature increased to 42° C. on adding the MNCB). The mixture was cooled down to 30° C., and the white precipitation was filtered with Kiriyama funnel having a 110 mmφ with a No. 5C filter paper, was immersed and washed with 100 mL of methanol, was immersed and washed with 200 mL of ion exchanged water, was dried under reduced pressure at 90° C., −0.1 MPa for 16 h and, thereby, to obtain a white powder with yield of 82% (weight 18.6 g), LC purity of 99.1%, and melting point (visual observation) of 160 to 161° C.. The product was [1,3-phenylenebis(propane-2,2-diyl)]bis(4,1-phenylene)bis(3-nitrobenzoate) represented by the above-described formula (k), hereinafter referred to as a dinitro compound 6.

TOF-MS (ESI): 643.209 (M−H)⁻

### Example 12

Synthesis of [1,3-phenylenebis(propane-2,2-diyl)]bis(4,1-phenylene)bis(3-aminobenzoate)

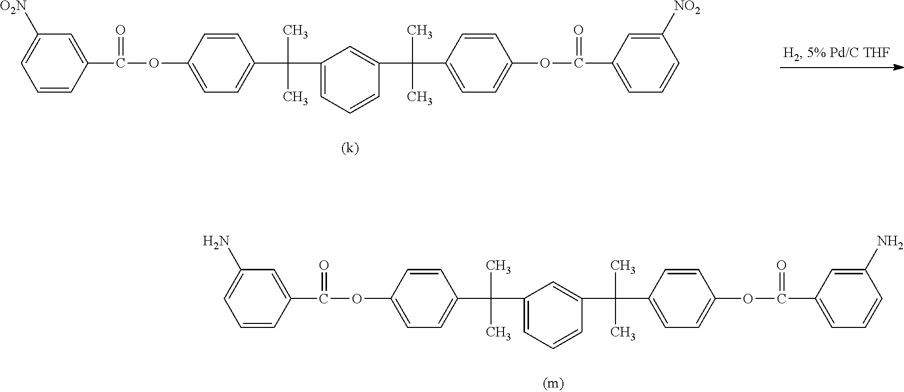 [SMILES: CC(C)(c1ccc(OC(=O)c2cccc([N+](=O)[O-])c2)cc1)c1cccc(C(C)(C)c2ccc(OC(=O)c3cccc([N+](=O)[O-])c3)cc2)c1 ; CC(C)(c1ccc(OC(=O)c2cccc(N)c2)cc1)c1cccc(C(C)(C)c2ccc(OC(=O)c3cccc(N)c3)cc2)c1]

17.0 g (26 mmol) of the dinitro compound 6, 120 mL of THE, and 0.1 g (as dry) of 5% Pd/C were prepared and sealed in a 300 mL autoclave. Four times of nitrogen substitution and four times of hydrogen substitution were repeated, the internal pressure of the autoclave was prepared to be 0.8 MPa and a leakage was confirmed, and after no hydrogen leakage was confirmed, a hydrogen introduction valve was closed and sealed. While being stirred at 200 rpm, the mixture was heated with a preheated heating mantle, the stirring rate was set to 1000 rpm at the timing where the temperature reached 60° C., the hydrogen introduction valve was opened and, thus, a hydrogenation reaction was started (this timing was set to reaction start 0 min). The reaction was performed at 65'1° C., under 0.80 MPa constant pressure. The reaction was performed until the instantaneous hydrogen absorption was no longer observed with a large flowmeter. At this time, the hydrogenation period was 28 min. The hydrogen introduction valve was closed, the mixture was stirred for 60 min, no drop in the internal pressure was confirmed and the stirring was stopped, after the hydrogen in the autoclave was exhausted, the nitrogen substitution (gauge pressure of 0 to 0.3 MPa) was performed for three times. The autoclave was opened, the catalyst was filtered, and the filtrate was cooled down to 5° C., but no crystal was deposited, and therefore, the solvent was distilled away (paste form) with an evaporator, 100 mL of methanol was added to cause a thermal dissolution, and the mixture was cooled down to 10° C. and, thus, a white powder was deposited. The mixture was filtered with Kiriyama funnel having 110 mmφ with a No.5C filter paper, was immersed and washed with 50 mL of methanol and 100 mL of ion exchanged water, was dried under reduced pressure at 90° C., −0.1 MPa for 16 h and, thereby, to obtain a white powder with yield of 98% (weight 15.0 g), LC purity of 99.5%, and melting point (visual observation) of 161 to 162° C. The product was [1,3-phenylenebis(propane-2,2-diyl)]bis(4,1-phenylene)bis(3-aminobenzoate) represented by the above-described formula (m).

TOF-MS (ESI): 585.276 (M+H)⁺

### Comparative Example 1

Synthesis of 1,4-bis(4-aminobenzoyloxy)benzene/hydroquinone-type p-diamine

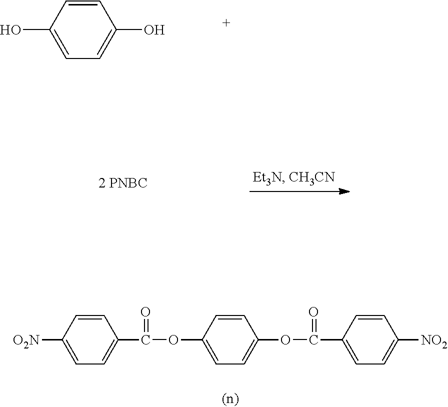 [SMILES: Oc1ccc(O)cc1 ; O=C(Oc1ccc(OC(=O)c2ccc([N+](=O)[O-])cc2)cc1)c1ccc([N+](=O)[O-])cc1]

In a 300 mL four-necked flask equipped with a mechanical stirrer and a thermometer, were placed 8.3 g (75 mmol) of hydroquinone, 200 mL of acetonitrile, and 16.0 g (158 mmol) of triethylamine, were heated to 45° C. and dissolved to obtain a reddish-brown transparent solution. To the solution, 25.0 g (158 mmol) of PNBC (m-nitrobenzoyl chloride) was added and, then, a white precipitation was immediately generated. The internal temperature increased from 45° C. to 68° C., and was cooled down after a short time to obtain light greenish white slurry. The mixture was stirred as it is at 45° C.for one hour, and the dissipation of the PNBC was confirmed by the HPLC. After the mixture was cooled down to a room temperature, the white precipitation was filtered out, and the cake was washed with methanol. The cake was air-dried and, thereby, to obtain a product with a white powder of 22.4 g/yield of 73%, mp of 263 to 264.5° C., and HPLC purity of 99.6%. The product was the compound represented by the above-described formula (n), hereinafter referred to as a dinitro compound 5.

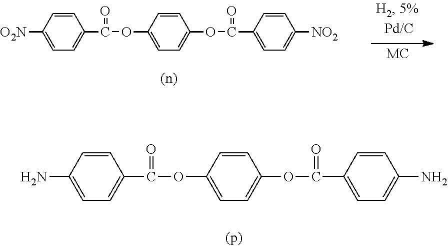

In a 300 mL SUS autoclave, were placed 22.5 g (53 mmol/purity conversion) of the above-described dinitro compound (n), 0.130 g (0.056 g as dry) of 5% Pd/C, and 150 mL of methyl cello solve (MC) and were sealed. Four times of nitrogen substitution and four times of hydrogen substitution were repeated, and no gas leakage was confirmed with a soap solution. The temperature of the mixture was increased to 70° C. at 150 rpm stirring under hydrogen 0.8 MPa constant pressure. The frequency of stirring was increased to 1000 rpm, and a hydrogen introduction valve was opened. While the internal temperature was kept at 85 to 90° C., a theoretical quantity of hydrogen was absorbed in 42 minutes, was aged for another 20 minutes, and it was confirmed that the internal pressure did not drop. After the nitrogen substitution, the autoclave was opened, and 1 L of DMF was added to white mousse-like slurry to be dissolved at a reflux temperature. The used catalyst was hot filtered, the filtrate was slowly cooled, and the generated precipitation was filtered out at 5° C. 200 mL of γ-butyrolactone was added to the cake, and they were heated to 165° C. to be dissolved. The mixture was slowly cooled down to 30° C., and the generated precipitation was filtered out. The cake was washed with methanol and air-dried and, thereby, to obtain a product with a pinkish white powder of 12.6 g/yield of 71%, mp of >300° C., and HPLC purity of 96%. The product was 1,4-bis(4-aminobenzoyloxy)benzene represented by the above-described formula (p).

### Comparative Example 2

Synthesis of 2,5-bis(4-aminobenzoyloxy)toluene/methylhydroquinone-type p-diamine

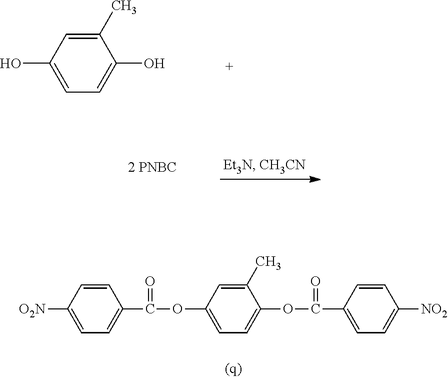

In a 300 mL four-necked flask equipped with a mechanical stirrer and a thermometer, were placed 9.3 g (75 mmol) of methylhydroquinone, 200 mL of acetonitrile, 16.0 g (158 mmol) of triethylamine and were dissolved at room temperature to obtain a light-yellow transparent solution. To the solution, 25.0 g (158 mmol) of PNBC (m-nitrobenzoyl chloride) is added and, then, a white precipitation was immediately generated. The internal temperature increased from 19° C. to 39° C., and was further heated to increase the temperature to 80° C. (white slurry). The mixture was stirred as it is for one hour, and the dissipation of the PNBC was confirmed by the HPLC. The mixture was let stand to cool down to a room temperature, the white precipitation was filtered out, and the cake was washed with methanol. The cake was air-dried and, thereby, to obtain a product with a white powder of 24.8 g/yield of 98%, mp of 269 to 270.5° C., and HPLC purity of 98%. The product was the compound represented by the above-described formula (q), hereinafter referred to as a dinitro compound 6.

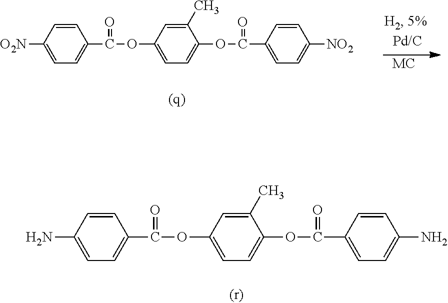

In a 300 mL SUS autoclave, were placed 10.6 g (25 mmol/purity conversion) of the above-described dinitro compound (q), 0.065 g (0.028 g as dry) of 5% Pd/C, and 180 mL of methyl cello solve (MC) and were sealed. Four times of nitrogen substitution and four times of hydrogen substitution were repeated, and no gas leakage was confirmed with a soap solution. The temperature of the mixture was increased to 70° C. at 150 rpm stirring under hydrogen 0.8 MPa constant pressure. The frequency of stirring was increased to 1000 rpm, and a hydrogen introduction valve was opened. While the internal temperature was kept at 90 to 95° C., a theoretical quantity of hydrogen was absorbed in 42 minutes, was aged for another 20 minutes, and it was confirmed that the internal pressure did not drop. After the nitrogen substitution, the autoclave was opened, and the used catalyst was hot-filtered. 45 mL of ion exchanged water was added (white slurry), and the mixture was heated to a reflux temperature and dissolved. The mixture was let stand to cool down to 20° C., the generated precipitation was filtered and dried and, thereby, to obtain a light-yellow powder of 7.5 g/yield of 83%, mp of 271.5 to 273° C., and LC purity of 96%. The product was 2,5-bis(4-aminobenzoyloxy)toluene represented by the above-described formula (r).

### Solubility of Diamine

Melting points and solubilities in various kinds of solvents of the diamines obtained in the above-described Examples and Comparative Examples are shown in Table 1 below. In the following Table 1, the evaluation +++ indicates soluble at room temperature, the evaluation ++ indicates soluble by heating, the evaluation + indicates half-soluble by heating, and the evaluation − indicates insoluble in solvent.

Among the para-type diamines, in particular, the unsubstituted hydroquinone-type p-diamine (melting point>300° C., Comparative Example 1) hot-dissolved only in DMF (N,N-dimethylformamide). The methylhydroquinone-type p-diamine (melting point 272 to 273° C., Comparative Example 2) having a methyl group in a center benzene ring also hot-dissolved barely in a high-polar solvent, such as MC (methyl cello solve) and DMSO (dimethylsulfoxide). In contrast, the meta-type diamines have the melting points that are each comparatively low and the solubilities to the various kinds of solvents that are also high. In particular, the bisphenol AF-type diamines easily dissolved in various solvents. Accordingly, the effects of the present invention were confirmed.

TABLE 1

| | Example 2 | Example 4 | Example 6 |
|---|---|---|---|
| | m-Diamine | | |
| X = | 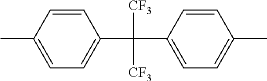 | 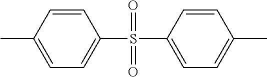 | 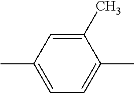 |
| Melting point, ° C. | 160-161 | 235-236 | 148-150 |
| Methanol | +++ | − | + |
| Isopropanol | ++ | − | + |
| MC | +++ | ++ | +++ |
| Acetonitrile | +++ | ++ | +++ |
| Acetone | +++ | ++ | +++ |
| Ethyl acetate | +++ | ++ | +++ |
| Chloroform | ++ | − | +++ |
| THF | +++ | + | +++ |
| Toluene | ++ | − | + |
| m-cresol | +++ | +++ | +++ |
| DMSO | +++ | +++ | +++ |
| DMF | +++ | +++ | +++ |
| | Example 8 | Com. Ex. 1 | Com. Ex. 2 |
| | m-Diamine | p-Diamine | |
| X = | 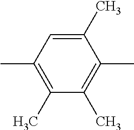 | 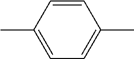 | 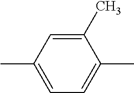 |
| Melting point, ° C. | 187-189 | >300 | 272-273 |
| Methanol | + | | − |
| Isopropanol | + | − | − |
| MC | +++ | − | ++ |
| Acetonitrile | +++ | − | + |
| Acetone | +++ | − | + |
| Ethyl acetate | +++ | − | + |
| Chloroform | +++ | − | + |
| THF | +++ | − | + |
| Toluene | + | − | + |
| m-cresol | +++ | − | + |
| DMSO | +++ | + | ++ |
| DMF | +++ | ++ | +++ |

Sample 30 mg/solvent 2 mL, the evaluation +++ indicates soluble at room temperature, the evaluation ++ indicates soluble by heating, the evaluation + indicates half-soluble by heating, and the evaluation − indicates insoluble.

### Example 13

### Synthesis of Polyimide by Polymerization Between Diamine Compound (Bisphenol AF Type M-Diamine, Represented by Formula (b)) Obtained in Example 2 With Pyromellitic Dianhydride (PMDA)

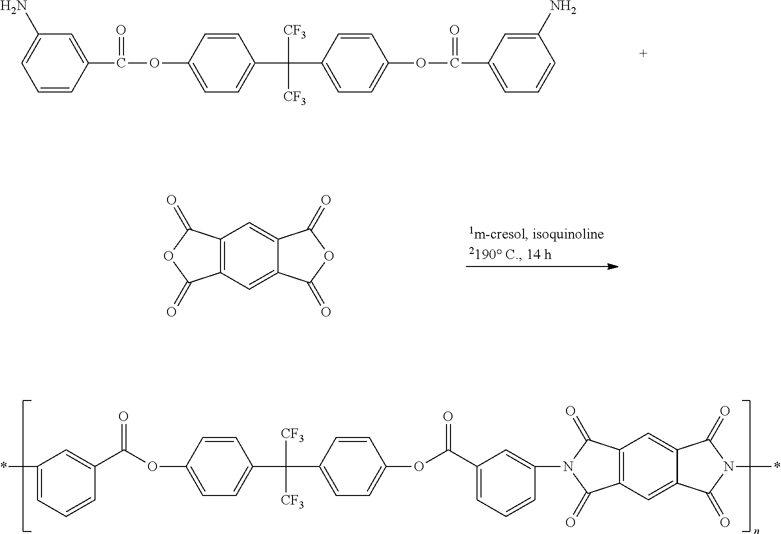

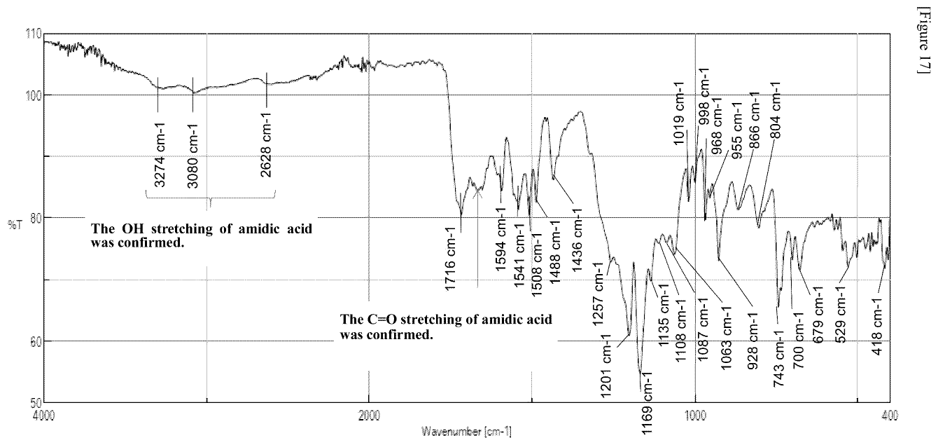

In a 100 mL separable flask equipped with a mechanical stirrer, a thermometer, and a condenser, were placed 3.78 g (6.58 mmol) of the diamine compound (bisphenol AF type m-diamine represented by formula (b)) obtained in Example 2 and 20 mL of m-cresol, and were dissolved at room temperature to obtain a golden viscous solution. Under a nitrogen stream, 1.44 g (6.60 mmol) of pyromellitic dianhydride (PMDA) was added to the solution, and the mixture was stirred for two hours as it is. While stirring, the frequency of stirring was increased from 300 rpm to 400 rpm, from 400 rpm to 500 rpm as the viscosity increased. 0.50 g (3.8 mmol) of isoquinoline was added, and the mixture was stirred for another four hours. 0.5 g of polymerization liquid was extracted and was poured over 30 mL of methanol. The generated white precipitation was filtered out and dried. By the FT-IR analysis, it was confirmed that the generation of polyamide acid. The result is shown in FIG. 17.

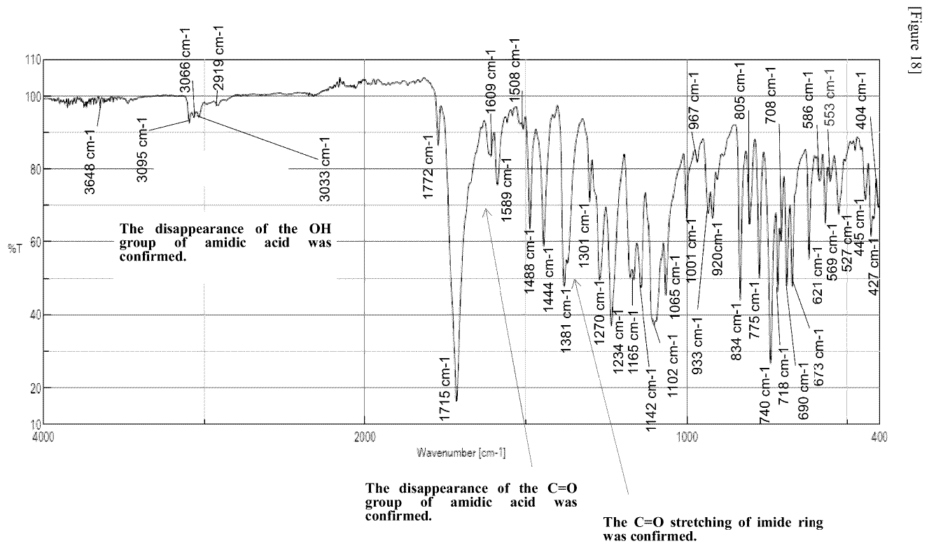

30 mL of m-cresol was added to the above-described viscous polymerization liquid, the mixture was heated to 190° C. and stirred for 14 hours. The mixture was let stand to cool down to a room temperature, and the polymerization liquid was poured over 300 mL of methanol. The generated precipitation was filtered out, the cake was washed with methanol, and was heated in a vacuum dryer (180° C./8 hours) and, thereby, to obtain 3.8 g of a yellow powder (yield 84%). By the FT-IR analysis, it is confirmed that polyimide could be synthesized. The result is shown in FIG. 18. The obtained polyimide was soluble in N-methylpyrrolidone (NMP) at room temperature.

### Example 14

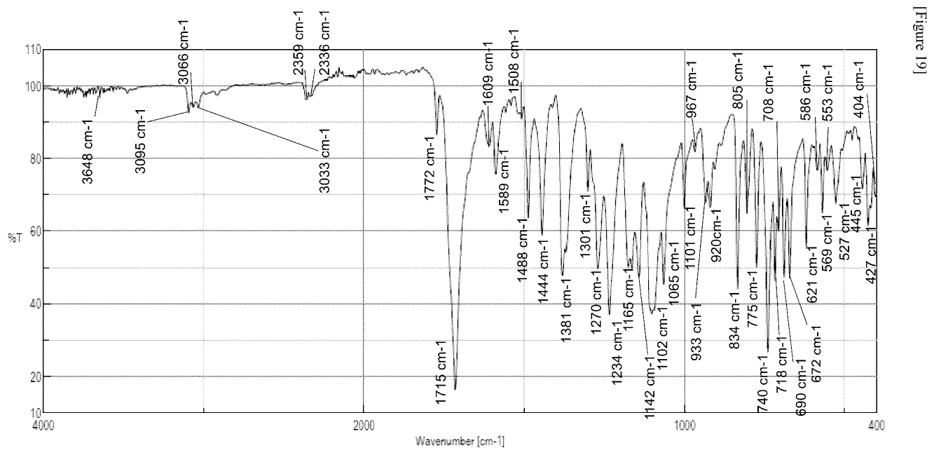

Example 9 was repeated except that PMDA was replaced to 4,4′-oxydiphthalic anhydride (ODPA) in Example 9, to synthesize a polyimide by polymerization between the diamine compound obtained in Example 2 and ODPA. The FT-IR spectrum of the obtained polyimide powder is shown in FIG. 19. The obtained polyimide was soluble in NMP at room temperature.

### Example 15

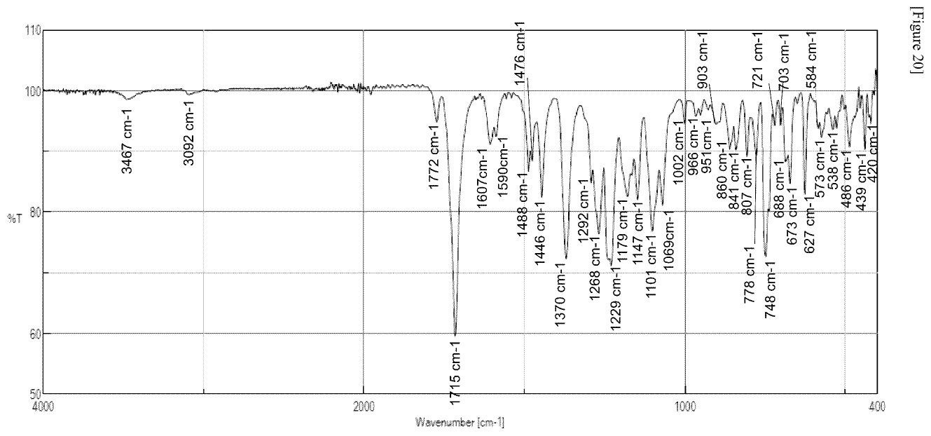

Example 9 was repeated except that PMDA was replaced to 4,4′-(hexafluoroisopropylidene)diphthalic anhydride (6FDA) in Example 9, to synthesize a polyimide by polymerization between the diamine compound obtained in Example 2 and 6FDA. The FT-IR spectrum of the obtained polyimide powder is shown in FIG. 20. The obtained polyimide was soluble in NMP at room temperature.

### Example 16

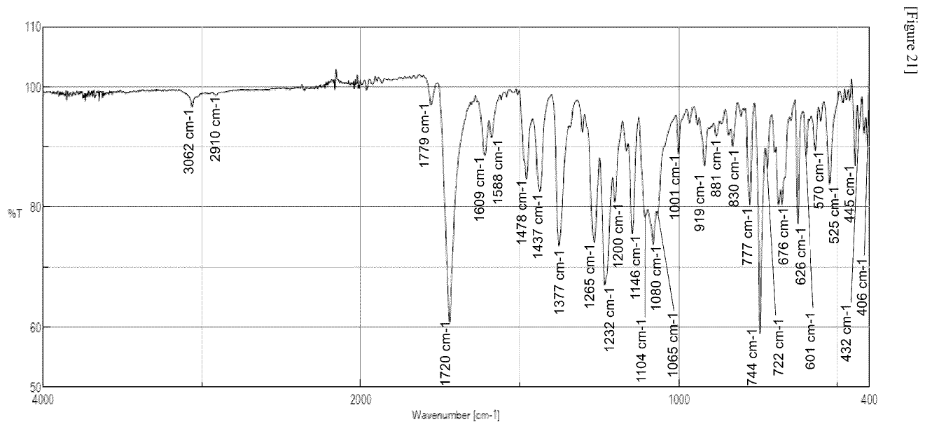

Example 9 was repeated except that the diamine compound obtained in Example 2 was replaced to the diamine compound obtained in Example 4 (bisphenol S type m-diamine) and PMDA was replaced to 4,4′-oxydiphthalic anhydride (ODPA) in Example 9, to synthesize a polyimide by polymerization between the diamine compound obtained in Example 4 and ODPA. The FT-IR spectrum of obtained polyimide powder is shown in FIG. 21. The obtained polyimide was soluble in NMP at room temperature.

### Example 17

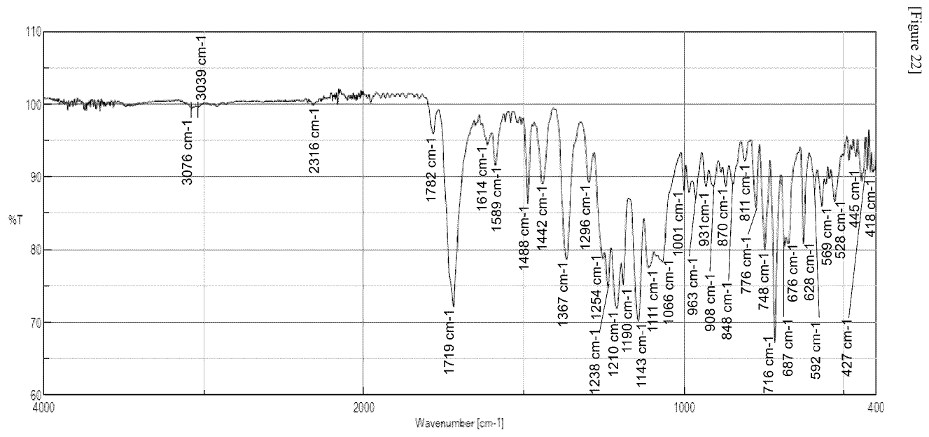

Example 9 was repeated except that the diamine compound obtained in Example 2 was replaced to the diamine compound obtained in Example 6 (methylhydroquinone-type m-diamine) and PMDA was replaced to 4,4′-oxydiphthalic anhydride (ODPA) in Example 9, to synthesize a polyimide by polymerization between the diamine compound obtained in Example 6 and ODPA. The FT-IR spectrum of the obtained polyimide powder is shown in FIG. 22. The obtained polyimide was soluble in NMP at room temperature.

### Example 18

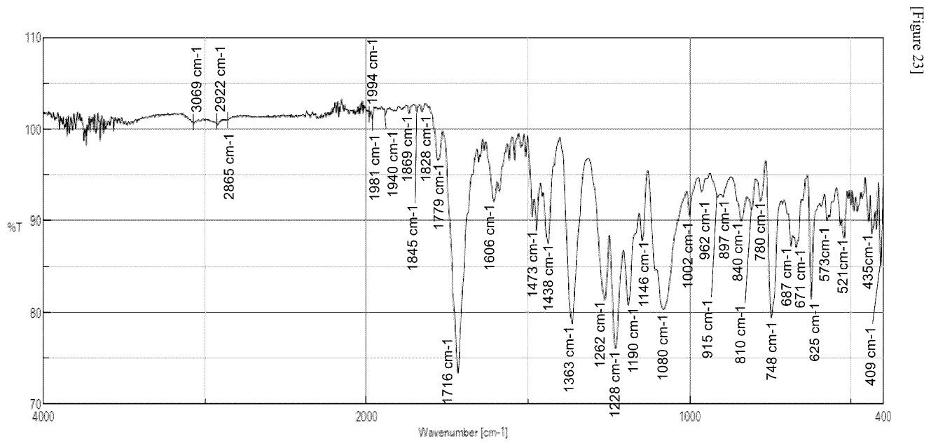

Example 9 was repeated except that the diamine compound obtained in Example 2 was replaced to the diamine compound obtained in Example 8 (trimethylhydroquinone-type m-diamine), and PMDA was replaced to 4,4′-oxydiphthalic anhydride (ODPA) in Example 9, to synthesize a polyimide by polymerization between the diamine compound obtained in Example 8 and the ODPA. The FT-IR spectrum of the obtained polyimide powder is shown in FIG. 23. The obtained polyimide was soluble in NMP at room temperature.

### INDUSTRIAL APPLICABILITY

The meta-type ester-containing aromatic diamines of the present invention are preferably usable as a novel polyimide raw material, enlarge possibility of the polyimide field introduced from the compounds, and are expected as a possible material having excellent high heat resistance and electric properties.

1. A compound represented by the following formula (1): 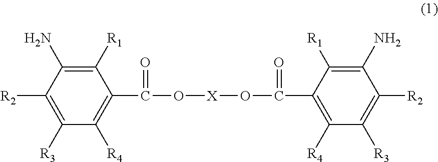
wherein X in formula (1) is selected from the following structures (a), (b) and (c), 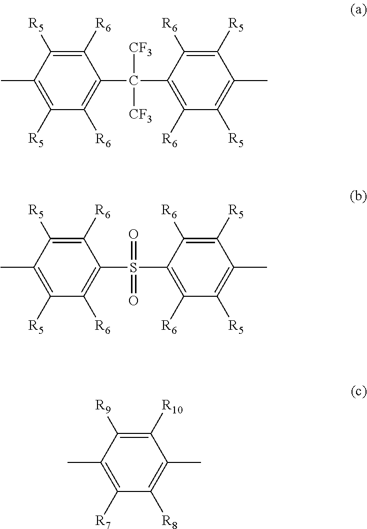
wherein R₁, R₂, R₃, and R₄ in the formula (1) and R₅, R₆, R₇, R₈, R₉ and R₁₀ in (a), (b) and (c) are, independently of each other, a hydrogen atom, an optionally substituted alkyl group having 1 to 6 carbon atoms, or an alkoxy group having 1 to 3 carbon atoms, provided that at least one of R₇, R₈, R₉, and R₁₀ is the alkyl group or the alkoxy group.

2. A compound represented by the following formula (1′): 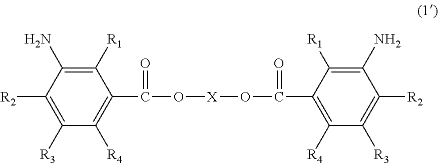
wherein X in formula (1) is the following structure (d), 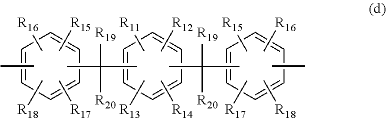
wherein R₁, R₂, R₃, R₄, R₁₁, R₁₂, R₁₃, R₁₄, R₁₅, R₁₆, R₁₇, R₁₈, R₁₉ and R₂₀ are, independently of each other, a hydrogen atom, an optionally substituted alkyl group having 1 to 6 carbon atoms, or an alkoxy group having 1 to 3 carbon atoms.

3. The compound according to claim 1, represented by the following formula (1a) or (1b): 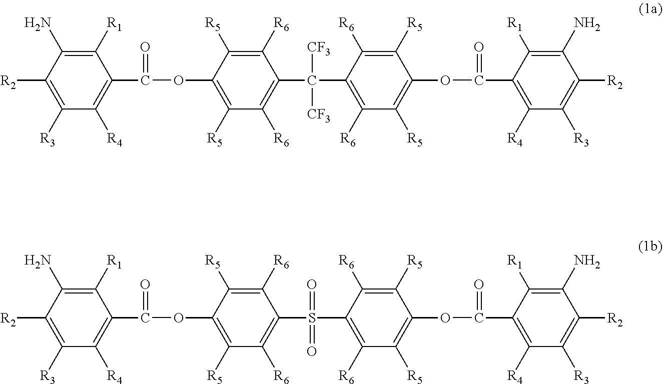
wherein R₁, R₂, R₃, R₄, R₅ and R₆ are as defined above.

4. The compound according to claim 3, wherein, in formula (1b), R₁, R₂, R₃, and R₄ are the hydrogen atom and R₅ and R₆ are, independently of each other, the hydrogen atom or the alkyl group having 1 to 6 carbon atoms.

5. The compound according to claim 1, represented by the following formula (1c): 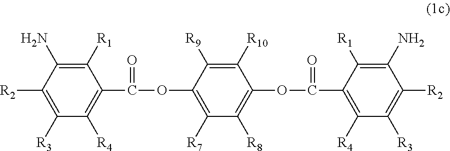
wherein R₁, R₂, R₃, R₄, R₇, R₈, R₉ and R₁₀ are as defined above.

6. The compound according to claim 5, wherein in formula (1c), R₁, R₂, R₃ and R₄ are the hydrogen atom, and R₇, R₈, R₉ and R₁₀ are, independently of each other, the hydrogen atom or the alkyl group having 1 to 6 carbon atoms, and at least one of R₇, R₈, R₉ and R₁₀ is the alkyl group.

7. The compound according to claim 2, wherein in formula (d), R₁, R₂, R₃ and R₄ are the hydrogen atom, and R₁₁, R₁₂, R₁₃, R₁₄, R₁₅, R₁₆, R₁₇, R₁₈, R₁₉ and R₂₀ are, independently of each other, the hydrogen atom or the alkyl group having 1 to 6 carbon atoms.

8. The compound according to claim 2 or 7, wherein X in formula (d) is selected from the following structures: 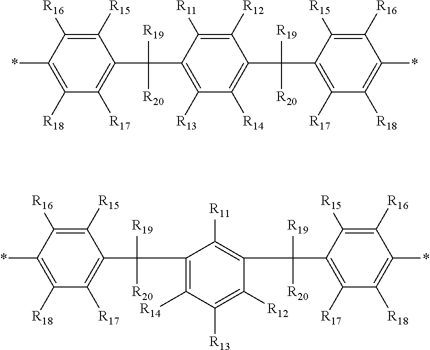
wherein R₁₁, R₁₂, R₁₃, R₁₄, R₁₅, R₁₆, R₁₇, R₁₈, R₁₉ and R₂₀ are as defined above, and the sites marked with * in the formulas bond to the oxygen atom.

9. A method for preparing a compound represented by the following formula (1): 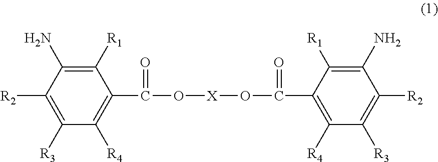
wherein X in formula (1) is selected from the following structures (a), (b) and (c), 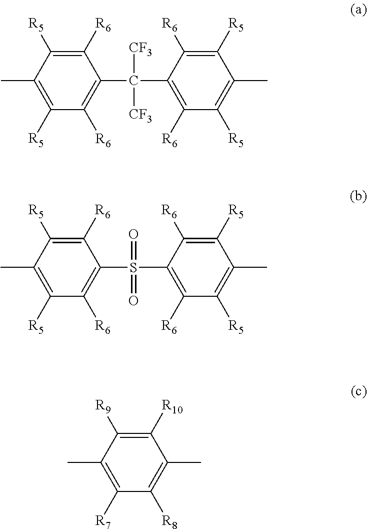
wherein R₁, R₂, R₃, and R₄ in the formula (1) and R₅, R₆, R₇, R₈, R₉, and R₁₀ in formulas (a), (b) and (c) are, independently of each other, a hydrogen atom, an optionally substituted alkyl group having 1 to 6 carbon atoms, or an alkoxy group having 1 to 3 carbon atoms, provided that at least one of R₇, R₈, R₉ and R₁₀ is the alkyl group or the alkoxy group,
wherein the method comprises a step of subjecting two nitro groups of the compound represented by the following formula (2) to reduction reaction: 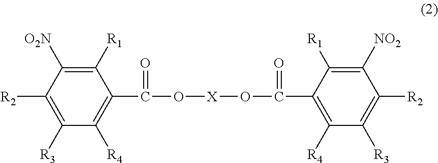
wherein R₁, R₂, R₃, R₄ and X are as defined above, to obtain the compound represented by the formula (1).

10. The method according to claim 9, wherein X is selected from the following structures (a) and (b): 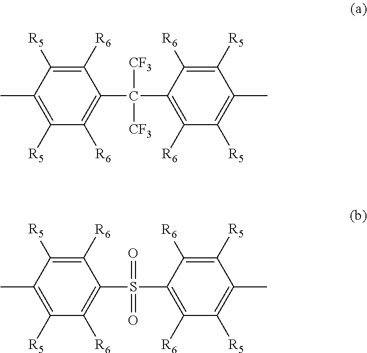
wherein R₁, R₂, R₃, R₄, R₅ and R₆ are as defined above.

11. The method according to claim 10, wherein R₁, R₂, R₃, and R₄ are the hydrogen atom, and R₅ and R₆ are, independently of each other, the hydrogen atom or the alkyl group having 1 to 6 carbon atoms.

12. The method according to claim 9, wherein X is the following formula (c): 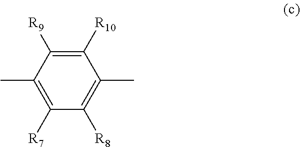
wherein R₁, R₂, R₃, R₄, R₇, R₈, R₉ and R₁₀ are as defined above.

13. The method according to claim 12, wherein R₁, R₂, R₃ and R₄ are the hydrogen atom, and R₇, R₈, R₉ and R₁₀ are, independently of each other, the hydrogen atom or the alkyl group having 1 to 6 carbon atoms, and at least one of R₇, R₈, R₉ and R₁₀ is the alkyl group.

14. A method for preparing a compound represented by the following formula (1′): 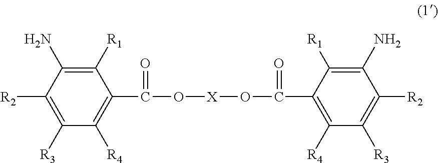
wherein X in formula (1′) is the following structure (d), 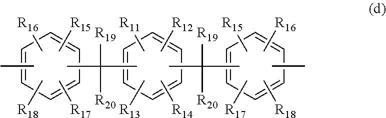
wherein R₁, R₂, R₃, R₄, R₁₁, R₁₂, R₁₃, R₁₄, R₁₅, R₁₆, R₁₇, R₁₈, R₁₉ and R₂₀ are, independently of each other, a hydrogen atom, an optionally substituted alkyl group having 1 to 6 carbon atoms, or an alkoxy group having 1 to 3 carbon atoms,
wherein the method comprising a step of subjecting two nitro groups of the compound represented by the following formula (2) to reduction reaction, 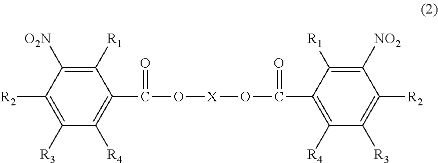
wherein R₁, R₂, R₃, R₄ and X are as defined above, to obtain the compound represented by the formula (1′).

15. The method according to claim 14, wherein R₁, R₂, R₃ and R₄ are the hydrogen atom, and R₁₁, R₁₂, R₁₃, R₁₄, R₁₅, R₁₆, R₁₇, R₁₈, R₁₉ and R₂₀ are, independently of each other, the hydrogen atom or the alkyl group having 1 to 6 carbon atoms.

16. The method according to claim 14 or 15, wherein X in formula (d) is selected from the following structures: 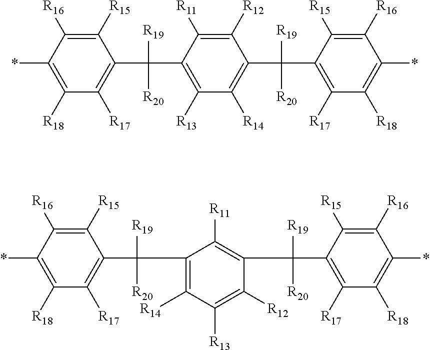
wherein R₁₁, R₁₂, R₁₃, R₁₄, R₁₅, R₁₆, R₁₇, R₁₈, R₁₉ and R₂₀ are as defined above, and the sites marked with * in the formulas bond to the oxygen atom.

17. A polyimide compound being a product of reaction of the compound according to any one of claims 1 to 8 with an acid anhydride.

18. The polyimide compound according to claim 17, wherein the acid anhydride is at least one selected from the group consisting of pyromellitic dianhydride, 3,3′,4,4′-biphenyltetracarboxylic dianhydride, 2,3,3′,4′-biphenyltetracarboxylic dianhydride, benzophenone-3,4,3′,4′-tetracarboxylic dianhydride, 4,4′-(2,2-hexafluoroisopropylidene) diphthalic dianhydride, 2,2-bis[3-(3,4-dicarboxyphenoxy) phenyl] propane dianhydride, 2,2-bis[4-(3,4-dicarboxyphenoxy) phenyl] propane dianhydride, and 3,3′,4,4′-diphenylsulfonetetracarboxylicdianhydride, and oxy-4,4′-diphthalic dianhydride.

19. The polyimide compound according to claim 17 or 18, having a number average molecular weight of 2,000 to 200,000.

20. A polyimide compound being a product of reaction of the compound according to any one of claims 1 to 8, an acid anhydride, with a diamine compound other than the compound according to claims 1 to 8, wherein the ratio of units derived from the compound according to any one of claims 1 to 8 is 10 mol % to 100 mol %, relative to the total moles of units derived from the compound according to any one of claims 1 to 8 and the diamine compound other than the compound according to claims 1 to 8.

21. A molded product comprising the polyimide compound according to any one of claims 17 to 20.
